(19) United States
(12) Patent Application Publication
(10) Pub. No.: US 2015/0342490 A1
(43) Pub. Date: Dec. 3, 2015

(54) BIOLOGICAL SIGNAL DETECTING APPARATUS AND IMPLANTABLE MEDICAL DEVICE

(71) Applicant: OLYMPUS CORPORATION, Tokyo (JP)

(72) Inventors: Shrestha Sachin, Leuven (BE); Refet Firat Yazicioglu, Leuven (BE); Torfs Tom, Leuven (BE); Masato Osawa, Tokyo (JP)

(73) Assignee: OLYMPUS CORPORATION, Tokyo (JP)

(21) Appl. No.: 14/824,260

(22) Filed: Aug. 12, 2015

Related U.S. Application Data
(63) Continuation of application No. PCT/JP2013/077189, filed on Sep. 30, 2013.

(30) Foreign Application Priority Data
Feb. 15, 2013 (EP) 13155548.4

(51) Int. Cl. A61B 5/04; A61B 5/00; A61N 1/365; A61B 5/0456

(52) U.S. Cl. CPC A61B 5/04014; A61B 5/0456; A61B 5/686; A61N 1/36514

(57) ABSTRACT

A biological signal detecting apparatus includes a BPF to which a biological signal is input via a lead directly connected to a subcutaneous tissue that emits the biological signal including a predetermined signal of a first frequency and which outputs a filtered biological signal by filtering a biological event signal of a predetermined frequency including the first frequency; and a peak detecting means to which at least the filtered biological signal is input and which detects a peak location of the biological event signal. The BPF includes a first order HPF which filters a frequency higher than a second frequency and a first order LPF which filters a frequency lower than a third frequency. The HPF and the LPF are connected in series between the lead and the peak detecting means. A difference between the second frequency and the third frequency is less than or equal to 10 Hz.

The present application is a continuous application based on a PCT International Application No. PCT/JP2013/077189, filed on Sep. 30, 2013, whose priority is claimed on European Patent Application No. 13155548.4, filed on February 15, 2013, the contents of both the PCT International Application and the European Patent Application are incorporated herein by reference.

### BACKGROUND OF THE INVENTION

1. Field of the Invention

The present disclosure relates generally to the field of biomedical devices and systems.

More specifically, the present invention relates to technology associated with an R-wave detector and an implantable medical device (IMD).

2. Description of Related Art

An electrical signal generated by cardiac activity is referred to as an intra-electrocardiogram (IECG) signal. In an IMD, the IECG is detected from a lead installed on a heart. In an extracorporeal biological signal detecting apparatus, the IECG propagating through a biological body is detected by installing an electrode on the skin.

When the heart works normally, the heart is in a state referred to as a normal sinus rhythm (NSR). Hereinafter, characteristics of the IECG signal in the NSR will be described using FIGS. 1 and 2. FIG. 1 is the citation from FIG. 4.4 of Design of Cardiac Pacemakers (edited by John G Webster, ISBN 0-7803-1134-5), and FIG. 2 is the citation from FIG. 8.6.

When the IECG in the NSR is measured, an IECG referred to as QRS waves as illustrated in FIG. 1 is obtained. The letters P, Q, R, 5, T, and U illustrated in FIG. 1 represent P, Q, R, S, T, and U waves, respectively.

Results of frequency analysis (Fourier transform) of the IECG in the NSR are illustrated in FIG. 2. As can be seen from a graph, the T wave is a signal having a spectrum center at about 5 Hz and the R wave is a signal having a spectrum center at about 10 Hz and spectrum spreading at about 6 Hz to about 30 Hz.

The symptom called a left bundle branch block may occur in patients to whom cardiac resynchronization therapy (CRT) is applicable. When the left bundle branch block occurs in a patient, an IECG signal IFCG from a left ventricle (LV) is delayed and detected with respect to an IECG signal IECG from a right ventricle (RV).

This phenomenon results in a phenomenon in which the LV is delayed and contracted with respect to the RV, and results in the function degradation of the LV for a long period of time.

It is possible to detect the symptom of the left bundle branch block by installing leads on both the LV and RV of such a patient and simultaneously monitoring the LV and RV.

The function degradation of the ventricle can be reduced by applying a pacing pulse for the CRT to the heart for the patient with the left bundle branch block. However, it is preferred that each of detection delays of a peak of R waves from the LV and a peak of R waves from the RV be 10 ms or less and that the LV is immediately paced when the peak of the R waves from the LV that is at least 10 ms later than the peak of the R waves from the RV has been detected in order to obtain the effect of an effective CRT.

When a heart normally operates, the heart enters a state called a sinus rhythm (NSR). Hereinafter, characteristics of the IECG in the NSR state will be described with reference to FIGS. 1 and 2. FIG. 1 shows a diagram cited from FIG. 4.4 of John G Webster, David M. Beans, “Design of Cardiac Pacemakers”, New York, IEEE press, 1995, pp.171-213., and FIG. 2 shows a diagram cited from FIG. 8.6 of John G Webster, David M. Beans, “Design of Cardiac Pacemakers”, New York, IEEE press, 1995, pp.171-213.

When an IECG in the NSR state is measured, an IECG called QRS waves as shown in FIG. 1 is obtained. FIG. 1 shows a waveform of the IECG. In this drawing, a horizontal direction represents time, and a vertical direction represents amplitude. Characteristics of P, Q, R, S, T, and U described in FIG. 1 represent signals called a P wave, a Q wave, an R wave, an S wave, a T wave, and a U wave in the IECG, respectively.

FIG. 2 shows results obtained by performing a frequency analysis (Fourier transform) of the IECG in the NSR state. From a graph in FIG. 2, it can be seen that the center of a spectrum of the T wave appears at approximately 5 Hz, and the center of a spectrum of the R wave appears at approximately 8 to 18 Hz.

Though the characteristic of the IECG is explained, extra-electrocardiogram (EECG) has the same characteristics as well. Hereinafter, IECG means intra-electrocardiogram and EECG means extra-electrocardiogram, and ECG means both IECG and EECG.

For a patient to whom an implantable Cardiac Resynchronization Therapy (CRT) is applied, a cardiac seizure called a Left bundle branch block (LBBB) may occur. When the LBBB occurs in a patient, the R-wave peak of the IECG from the Left Ventricle (LV) is delayed with respect to the R-wave peak of the IECG from the Right Ventricle(RV). This seizure causes the contraction delay of the LV with respect to RV that may result in the deterioration of left ventricular function.

LBBB can be treated by pacing the LV when the delay of the R-wave peak from LV with respect to RV is seen.

To obtain the effective CRT result, it is desirable to detect R-wave peak of LV and RV less than 10 ms delay respectively.

Hereinafter, description will be made with respect to a structure in which the Implantable Cardioverer Difibrillator (ICD) described in U.S. Pat. No. 5,891,169 detects a cardiac beat with reference to FIGS. 3 and 4. FIG. 3 shows a diagram corresponding to FIG. 2 of U.S. Pat. No. 5,891,169, and FIG. 4 shows a diagram corresponding to FIG. 3 of U.S. Pat. No. 5,891,169.

Note that CRT apparatus is detecting the R-wave peak of the IECG as the same manner of the ICD.

FIG. 3 illustrates a method of measuring a cardiac rate using a fixed threshold value. In this drawing, a horizontal direction represents time, and a vertical direction represents amplitude. In this drawing, a solid line represents an IECG, and a broken line represents a threshold value. A portion at which the solid line and the broken line intersect each other is counted as a cardiac beat. The solid line in FIG. 3(a) represents an IECG of a typical NSR, the solid line in FIG. 3(b) represents an IECG of a typical ventricular tachycardia (VT), and the solid line in FIG. 3(c) represents an IECG of a typical VF. In addition, broken lines TI, TII, and TIII represent threshold values that are very suitable to detect cardiac beats of the NSR, VT, and VF, respectively.

In general, when the counted cardiac rate is 145 bpm (beats per minute) or less, it is diagnosed as SNR, when the counted cardiac rate is 146 to 225 bpm, it is diagnosed as VT, and when the counted cardiac rate is 226 bpm or more, it is diagnosed as VF. In addition, when peak values of IECGs of the NSR, VT, and VF are compared to each other, the peak value of the VF apparently has a value smaller than those of the NSR and VT. As can be seen from FIG. 3, when a high threshold value, which is very suitable to detect the cardiac beat of the NSR and VT, is set, the cardiac beat of the VF is not detected, and when a low threshold value, which is very suitable to detect the cardiac beat of the VF, is set, there is a danger that the T wave that is present in the IECG of the NSR and VT is detected as the cardiac beat.

Therefore, as indicated by a broken line in FIG. 4(a), a method of detecting the cardiac beat using a threshold value that exponentially attenuates from a peak position of the R wave (the solid line at an upper end of FIG. 4) is generally used. This method is called an AGC method. FIG. 4 illustrates a method of measuring the cardiac rate using the AGC method. A solid line in FIG. 4(a) represents an IECG, and a broken line in FIG. 4(a) represents a threshold voltage in the AGC method. In the drawing, a horizontal direction represents time, and a vertical direction represents amplitude. In a general AGC method, the threshold value has a characteristic of exponentially decreasing from a 75% value of a peak value when detecting the R wave, and a time constant thereof is 400 ms.

A bar graph of FIG. 4(b) represents a timing at which a cardiac beat is detected, and a portion of a range A in FIG. 4(b) corresponds to the NSR, a portion of a range B corresponds to the VT, and a portion of a range C corresponds to the VF. As can be seen from this drawing, the cardiac rate in NSR and VT sections is correctly detected.

Since threshold value intersects at the rising slope of the R-wave at IECG, AGC method can always detect the R-wave less than 10 ms delay with respect to the actual timing of the R-wave peak.

However, there is a case the R-wave of the patients who has a large T-wave amplitude cannot be detected properly. This case is described in FIG. 5.

A solid line in FIG. 5(a) represents an IECG, and a broken line in FIG. 5(a) represents a threshold voltage in the AGC method in the case of a large T-wave amplitude. In this case, the threshold voltage is crossing T-wave peak as well as R-wave peak.

As shown in FIG. 5(b), the heart beat is over counted at T-wave peak.

A methodology shown in IEEE JOURNAL OF SOLID-STATE CIRCUITS, VOL. 46, NO. 1, JANUARY 2011 can solve the problem shown in FIG. 5. FIG. 6 shows a diagram cited from FIG. 9 of IEEE JOURNAL OF SOLID-STATE CIRCUITS, VOL. 46, NO. 1, JANUARY 2011, and FIG. 7 shows a diagram cited from FIG. 11 of IEEE JOURNAL OF SOLID-STATE CIRCUITS, VOL. 46, NO. 1, JANUARY 2011.

An IECG input from IN+ and IN− pin in FIG. 6 is filtered at Switched Capacitor Low-Pass Filter (SCLPF) block to extract the frequency component of R-wave. A SCLPF in FIG. 6 has a 6th order band-pass filter characteristics the center frequency of which is located at 16 Hz. A transfer function in the right bottom picture of the FIG. 7 corresponds to it.

Thanks to the sharp cut-off characteristic of the 6th order band-pass filter, T-wave wave form which has the spectrum around 5 Hz is effectively eliminated. By squaring and adding filtered signal (BPI and BPQ), BPI2 +BPQ2 is obtained. This wave form is shown at the top of the FIG. 7. Since T-wave form is completely suppressed at BPI2 +BPQ2 waveform, T-wave cover counting does not occur in this methodology.

Though this operation enable us to find R-wave peak more precisely, the 6th order band-pass filter which causes lagging of signal more than 10 ms cannot be suitable for CRT requirement.

There is a real-time QRS detection algorithm developed by Pan and Tompkins known as the algorithm that has the highest accuracy of the R-peak detection. FIG. 7 shows a diagram cited from FIG. 12.9 of “Biomedical Digital Signal Processing: C-Language Examples and Laboratory Experiments for the IBM Pc/Book and Disk”, Editor : Willis J. Tompkins, Publisher: Prentice Hall (1993 Mar. 2) , ISBN-10: 0130672165.

A real-time QRS detection algorithm developed by Pan and Tompkins was further described by Hamilton and Tompkins. It recognizes QRS complexes based on analyses of the slope, amplitude, and width.

As show in FIG. 7, this algorithm uses, differentiated IECG x[n], band-passed IECG y[n] and time-averaged IECG z[n].

Since this algorithm requires average operation of 32 data samples to obtain time-averaged IECG z[n], this algorithm also cannot meet the requirement of 10 ms delay for CRT operation because normal implantable medical devices has the sampling rate less than 1 ksps to reduce the power consumption. This means that at least 32 ms delay is caused in CRT apparatus the sampling rate of which is 1 ksps.

Hereinafter, a mechanism in which an implantable cardioverter defibrillator (ICD) disclosed in U.S. Pat. No. 5,891,169 detects beats of the heart will be described using FIGS. 3 and 4. FIG. 3 is an excerpt from FIG. 2 of U.S. Pat. No. 5,891,169, and FIG. 4 is an excerpt from FIG. 3 of U.S. Pat. No. 5,891,169.

FIG. 3 is a diagram illustrating a method of measuring a heart rate using a fixed threshold value, and an intersection portion between a solid line and a dotted line is counted as the heartbeat. The solid line on the top of FIG. 3 represents an IFCG of a typical NSR, the solid line on the middle of FIG. 3 represents an IECG of typical ventricular tachycardia (VT), and the solid line on the bottom of FIG. 3 represents an IECG of typical ventricular fibrillation (VF). In addition, dotted lines TI, TII, and TIII represent threshold values preferred for heartbeat detection of the NSR, the VT, and the VF, respectively.

In general, a counted heart rate of up to 145 beats per minute (bpm) is diagnosed as the NSR, a counted heart rate of 146 to 225 bpm is diagnosed as the VT, and a counted heart rate from 226 bpm is diagnosed as the VF.

As can be seen from FIG. 3, it is difficult to detect heartbeats of the VF when a high threshold value preferred to detect heartbeats of the NSR and the VT is set, and there is a risk of T waves in IECGs of the NSR and the VT being detected as heartbeats when a low threshold value preferred to detect heartbeats of the VF is set.

Because of this, a method of detecting heartbeats using a threshold value which is exponentially decayed from a peak location of the R wave (the solid line on the top of FIG. 4) as indicated by the dotted line on the top in FIG. 4 is generally used. This method is referred to as an automatic gain control (AGC) scheme. FIG. 4 is a diagram illustrating a method of measuring a heart rate using the AGC scheme. The solid line on the top of FIG. 4 represents an IECG, and the dotted line on the top of FIG. 4 represents a threshold voltage in the AGC scheme. In a general AGC scheme, there is a characteristic that a threshold value is exponentially decayed from 75% of a peak value when the R wave is detected, and its time constant is 400 ms.

A bar graph of the lower portion of FIG. 4 illustrates a timing at which the heartbeat has been detected, and a portion A illustrated in the lower portion of FIG. 4 corresponds to the NSR, a portion B corresponds to the VT, and a portion C corresponds to the VF.

As can be seen from FIG. 4, the heart rate is accurately detected in NSR and VT intervals. Because the threshold voltage intersects at a rising portion of the R wave and a detection delay of the R wave in the AGC scheme is also constantly less than 10 ms, an effective CRT can also be executed. On the other hand, in the VF interval, it is difficult to detect a peak of a small amplitude following a large amplitude and VF detection may be delayed.

Further, as illustrated in FIG. 5, it may be difficult to accurately detect the R wave of the patient having the large T wave in the AGC scheme. The solid line of FIG. 5(a) represents an IECG signal IECG, and the dashed line represents a threshold voltage of the AGC scheme. A vertical bar of FIG. 5(b) represents a position of the R wave of an IECG signal IECG detected by the AGC scheme. In this case, the threshold voltage intersects a vertex of the T wave as well as a vertex of the R wave. As illustrated in FIG. 5(b), the heartbeat is over-counted at the location of the T wave.

A method disclosed in IEEE JOURNAL OF SOLID-STATE CIRCUITS, VOL. 46, NO. 1, JANUARY 2011 solves a problem of over-counting described in FIG. 5. FIG. 6 is a citation from FIG. 9 of IEEE JOURNAL OF SOLID-STATE CIRCUITS, VOL. 46, NO. 1, JANUARY 2011, and FIG. 7 is a citation from FIG. 11 of IEEE JOURNAL OF SOLID-STATE CIRCUITS, VOL. 46, NO. 1, JANUARY 2011.

As illustrated in FIG. 6, an analog signal processor constituting a biological signal detecting apparatus disclosed in IEEE JOURNAL OF SOLID-STATE CIRCUITS, VOL. 46, NO. 1, JANUARY 2011 filters only a frequency component of the R wave from the IECG signal IECG input between terminals IN+ and IN− using a switched capacitor low-pass filter (SCLPF), and strongly suppresses a signal having a frequency unnecessary for peak detection of the R wave such as the T wave. A transfer function of a sixth-order band pass filter (BPF) having the center frequency at 16 Hz is illustrated on the lower right of FIG. 7.

Each of waveforms of signals BPI and BPQ obtained by filtering the IECG signal IECG using the SCLPF is illustrated on the lower right of FIG. 7. As can be seen from the waveform, the waveform of a T wave part included in the IECG signal IECG according to an operation of the SCLPF is suppressed.

In addition, as can be seen from a square sum of BPI and BPQ and a waveform of BPI² BPQ² (see the upper graph of FIG. 7), the waveform of the T wave part is more strongly suppressed by a square-sum calculation performed within the DSP of FIG. 6, and the suppression contributes to more reliable detection of the R wave.

However, because transfer delay characteristics of this filter for equivalently implementing a sixth-order band pass characteristic using a third-order low-pass filter (LPF) are greater than 10 ms, there is a problem in that it is difficult to detect the R wave within a time necessary to perform effective CRT.

### SUMMARY OF INVENTION

According to a first aspect of the present invention, a biological signal detecting apparatus includes a BPF to which a biological signal is input via a lead directly connected to a subcutaneous tissue that emits the biological signal including a predetermined signal of a first frequency and which outputs a filtered biological signal by filtering a signal (biological event signal) of a predetermined frequency including the first frequency; and a peak detecting means to which at least the filtered biological signal is input and which detects a peak location of the biological event signal by processing the filtered biological signal, wherein the BPF includes a first order high-pass filter (HPF) which filters a frequency higher than a second frequency and a first order LPF which filters a frequency lower than a third frequency, wherein the HPF and the LPF are connected in series between the lead and the peak detecting means, and wherein a difference between the second frequency and the third frequency is less than or equal to 10 Hz.

According to a second aspect of the present invention, in the biological signal detecting apparatus according to the first aspect, the second frequency may be less than the first frequency and greater than or equal to 6 Hz, and the third frequency may be greater than the first frequency and less than or equal to 25 Hz.

According to a third aspect of the present invention, the biological signal detecting apparatus according to the first aspect may further include a gain amplifier which is connected between the HPF and the LPF and amplifies a signal gain.

According to a fourth aspect of the present invention, in the biological signal detecting apparatus according to the first aspect, the gain amplifier may be a variable gain amplifier, and a gain of the variable gain amplifier may be varied to an extent to which a signal component decayed by the BPF is compensated for.

According to a fifth aspect of the present invention, in the biological signal detecting apparatus according to the first aspect, both the HPF and the LPF may be configured in a Butterworth type.

According to a sixth aspect of the present invention, in the biological signal detecting apparatus according to the first aspect, both the HPF and the LPF may be configured in a switched capacitor type.

According to a seventh aspect of the present invention, an IMD includes the biological signal detecting apparatus according to the first aspect; a diagnosis means; and a therapy signal generating means, wherein the biological signal is an IECG signal, wherein the tissue is a heart, wherein the biological event signal is an R wave, wherein the lead is connected to at least one of an RV or an LV of the heart, wherein the diagnosis means receives an input of the peak location and outputs a diagnosis result corresponding to information of the peak location; and wherein the therapy signal generating means receives an input of the diagnosis result and applies a therapy signal corresponding to the diagnosis result to a ventricle for which therapy is necessary via the lead.

### BRIEF DESCRIPTION OF DRAWINGS

FIG. 1 shows a waveform of the IECG.

FIG. 2 shows results obtained by performing a frequency analysis (Fourier transform) of the IECG in the NSR state.

FIG. 3 illustrates a method of measuring a cardiac rate using a fixed threshold value.

FIG. 4 is a diagram illustrating a method of measuring a heart rate using the AGC scheme.

FIG. 5 describes a case the R-wave of the patients who has a large T-wave amplitude cannot be detected properly.

FIG. 6 shows a diagram cited from FIG. 9 of IEEE JOURNAL OF SOLID-STATE CIRCUITS, VOL. 46, NO. 1, JANUARY 2011.

FIG. 7 shows a diagram cited from FIG. 11 of IEEE JOURNAL OF SOLID-STATE CIRCUITS, VOL. 46, NO. 1, JANUARY 2011.

FIG. 8 is a diagram illustrating a relationship between a configuration of a biological signal detecting apparatus BIO_DETECT_APPARATUS according to each embodiment of the present invention and a biological body BDY serving as an implantation target of the biological signal detecting apparatus BIO_DETECT_APPARATUS.

FIG. 9A is a Bode diagram of the HPF.

FIG. 9B is a Bode diagram of the LPF.

FIG. 9C is a Bode diagram of the BPF.

FIG. 10A is a graph representing amplitude-frequency characteristics of each signal constituting a biological signal BIOG wherein the vertical axis represents amplitude and the horizontal axis represents frequency.

FIG. 10B is a graph illustrating amplitude-frequency characteristics of each signal constituting a filtered biological signal BP_BIO, wherein the vertical axis represents amplitude and the horizontal axis represents frequency.

FIG. 11 is a diagram illustrating a relationship between a configuration of the IMD according to each embodiment of the present invention and a heart HEART to be treated by the IMD.

FIG. 12 is a diagram illustrating a configuration of the biological signal detecting apparatus BIO_DETECT_APPARATUS according to this embodiment.

FIG. 13 is a diagram describing HPF in further detail.

FIG. 14 is a diagram describing LPF in further detail.

FIG. 15 is the MATLAB simulation result of the transfer function, in a linear phase band pass filter configuration consisting of a 1st order Butterworth HPF and 1st order Butterworth LPF.

FIG. 16A is a diagram illustrating relationships of frequencies and amplitudes of T, R, and P waves and EMI constituting an IECG signal IECG created based on K. A. Ellenbogen, G. N. Kay, C-P. Lau, and B. L. Wilkoff, “Clinical Cardiac Pacing, Defibrillation, and Resynchronization Therapy,” Elsevier, 3rd edition, 2007, ISBN 1-4160-2536-7.

FIG. 16B is a diagram illustrating a relationship between a frequency and amplitude of each signal constituting the filtered biological signal BP_BIO obtained according to a filtering operation of the BPF.

FIG. 17 is a diagram describing details of the peak detecting means PK_DETECT disclosed in this second embodiment.

FIG. 18 is a diagram describing operations of the heart HEART, the BPF, and the square calculation means SQR.

FIG. 19 is a diagram describing operations of the threshold generating means TH_GEN and the comparison means CMP.

FIG. 20 is a diagram describing operations of the polarity determination means P_DET and the peak search means P_SEARCH.

FIG. 21 illustrates simulation results representing a state of peak location detection when a representative NSR waveform was input to the biological signal detecting apparatus PK_DETECT_{—} APPARATUS, wherein the vertical axis represents amplitude, the horizontal axis represents time, a mark O represents a detected location of the R wave, a mark X represents a location of the R wave marked by a doctor, and a solid line represents an IECG signal IECG.

FIG. 22 illustrates a state of R-wave detection in a representative VT waveform.

FIG. 23 illustrates a state of R-wave detection in a representative VF waveform.

FIG. 24 illustrates different NSR waveforms of 5 patients were extracted and a result obtained by generating a histogram from a location of the P wave marked by the doctor and a deviation amount (t_{delay} of FIG. 20) of the timing of POW_{MAX}_{_{—}}_{FIN}.

FIG. 25 illustrates results of simulation of R-wave detection precision performed using an IECG signal obtained by intentionally superimposing a disturbance signal (noise) on the IECG signal IECG.

FIG. 26A is a graph for a comparison between a frequency component of a biological signal to be detected by the EECG signal detecting apparatus and a frequency component of a biological signal BIOG to be detected by an IECG signal detecting apparatus.

FIG. 26B is a graph for a comparison between a frequency component of a biological signal to be detected by the EECG signal detecting apparatus and a frequency component of a biological signal BIOG to be detected by an IECG signal detecting apparatus.

FIG. 27 illustrates a modified examples in which a variable gain amplifier PGA is inserted between the HPF and the LPF constituting the BPF.

FIG. 28 describing a case in which after a signal generated from a tissue ORG is amplified by an instrumentation amplifier (IA) via the lead LEAD, the amplified signal is input to the BPF and the peak detecting means PK_DETECT.

FIG. 29 is a flowchart illustrating another exemplary embodiment of the present invention.

### DETAILED DESCRIPTION OF THE INVENTION

### First Embodiment

### [Biological Signal Detecting Apparatus]

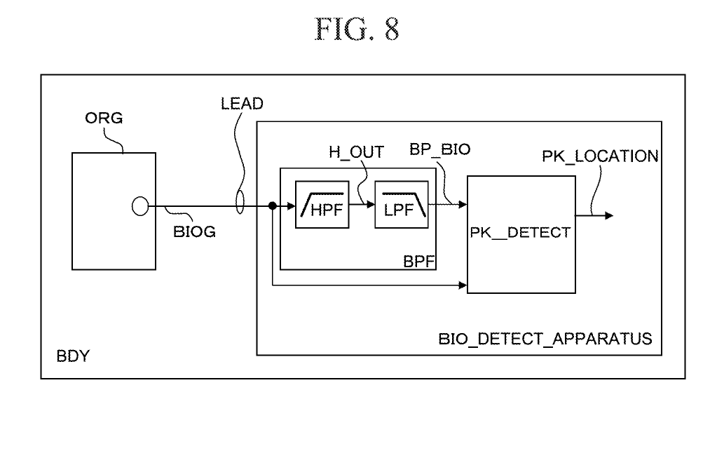

Hereinafter, the first embodiment of the present invention will be described with reference to FIG. 8. FIG. 8 is a diagram illustrating a relationship between a configuration of a biological signal detecting apparatus BIO_DETECT_APPARATUS according to each embodiment of the present invention and a biological body BDY serving as an implantation target of the biological signal detecting apparatus BIO_DETECT_APPARATUS.

First, the biological body BDY will be described. The biological body BDY has a tissue ORG, and the biological signal detecting apparatus BIO_DETECT_APPARATUS is implanted under the skin.

Next, the biological signal detecting apparatus BIO_DETECT_APPARATUS will be described. The biological signal detecting apparatus BIO_DETECT_APPARATUS includes a BPF and a peak detecting means PK_DETCT.

The BPF includes an HPF and an LPF.

The biological signal detecting apparatus BIO_DETECT_APPARATUS is connected to the tissue ORG via the lead LEAD. The HPF is connected to the lead LEAD and the LPF. The LPF is connected to the HPF and the peak detecting means PK_DETECT. The peak detecting means PK_DETECT is connected to the lead LEAD and the LPF.

A biological signal BIOG from the tissue ORG is input to the biological signal detecting apparatus BIO_DETECT_APPARATUS via the lead LEAD.

The biological signal BIOG including a first frequency fl is input to the HPF, and a frequency component of a second frequency f2 or less included in the input biological signal BIOG is decayed and output as a high-pass output H_OUT.

The high-pass output H_OUT is input to the LPF, and a frequency component of a third frequency f3 or more included in the input high-pass output H_OUT is decayed and output as a filtered biological signal BP_BIO.

The biological signal BIOG and the filtered biological signal BP_BIO are input to the peak detecting means PK_DETECT. The peak detecting means PK_DETECT detects a peak location of the biological signal BIOG with high precision by processing the filtered biological signal BP_BIO after suppressing a frequency component other than the first frequency f1, and outputs the detected peak location as a peak location PK_LOCATION.

### [Cancellation of Signal Delay]

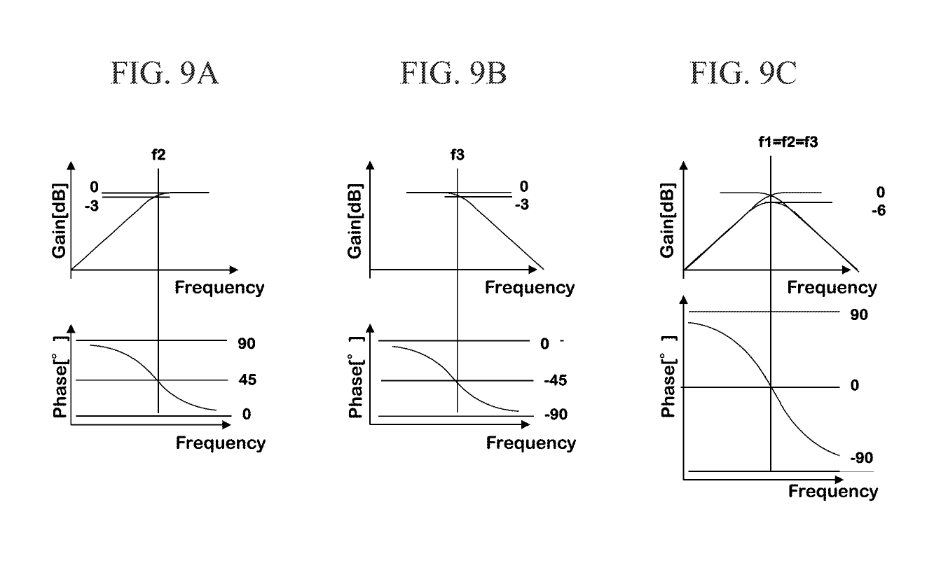

Hereinafter, a mechanism in which a signal delay is substantially canceled in a pass band of the BPF disclosed in this example embodiment will be described using FIGS. 9A to 9C.

FIG. 9A is a Bode diagram of the HPF, FIG. 9B is a Bode diagram of the LPF, and FIG. 9C is a Bode diagram of the BPF.

Each of the upper graphs of FIGS. 9A to 9C represents gain-frequency characteristics of a filter, wherein the horizontal axis represents a frequency (logarithmic expression) and the vertical axis represents a gain. Each of the lower graphs of FIGS. 9A to 9C represents frequency-phase characteristics of a filter, wherein the horizontal axis represents a frequency (logarithmic expression) and the vertical axis represents a phase lead (a positive is a phase lead and a negative is a phase lag).

First, the gain-frequency characteristics of the HPF will be described using the upper diagram of FIG. 9A. The HPF is a first order filter which decays a signal at a ratio of 20 dB/DEC at a frequency lower than the cutoff frequency. A decay rate of a signal at the cutoff frequency is 3 dB, and a decay rate of a signal at a frequency higher than the cutoff frequency is 0 dB. The cutoff frequency of this filter is set to the second frequency f2.

Next, the phase-frequency characteristics of the HPF will be described using the lower diagram of FIG. 9A. The phase lead of the HPF gradually approaches 90° at a low frequency. The phase lead at the cutoff frequency is 45° and gradually approaches 0° at a high frequency.

Next, the gain-frequency characteristics of the LPF will be described using the upper diagram of FIG. 9B. The LPF is a first order filter which decays a signal at a ratio of 20 dB/DEC at a frequency higher than the cutoff frequency. A decay rate of a signal at the cutoff frequency is 3 dB, and a decay rate of a signal at a frequency lower than the cutoff frequency is 0 dB. The cutoff frequency of this filter is set to the third frequency f3.

Next, the phase-frequency characteristics of the LPF will be described using the lower diagram of FIG. 9B. The phase lead of the LPF gradually approaches 0° at a low frequency. The phase lead at the cutoff frequency is −45° and gradually approaches 90° at a high frequency.

Finally, the BPF will be described.

First, the gain-frequency characteristics will be described using the upper diagram of FIG. 9C. Because this BPF is implemented by connecting the HPF and the LPF in series, the gain-frequency characteristics are represented by superimposing the upper graph of FIG. 9A and the upper graph of FIG. 9B.

In this embodiment, because the cutoff frequency f2 of the HPF is consistent with the cutoff frequency f3 of the LPF, the decay rate of the signal at the center frequency is 6 dB. At a frequency lower than the center frequency, the signal is decayed at a ratio of 20 dB/DEC. At a frequency higher than the center frequency, the signal is decayed at a ratio of 20 dB/DEC.

Then, the gain-frequency characteristics will be described using the lower diagram of FIG. 9C.

Because this BPF is implemented by connecting the HPF and the LPF in series, the phase-frequency characteristics are represented by superimposing the lower graph of FIG. 9A and the lower graph of FIG. 9B.

When the cutoff frequency of the HPF is consistent with the cutoff frequency of the LPF, the phase lead at the center frequency is 0°. At a frequency lower than the center frequency, the phase lead gradually approaches 90°. At a frequency higher than the center frequency, the phase lead gradually approaches −90°.

Because the center frequency of the BPF is set to the first frequency f1, a biological signal having the first frequency is decayed by 6 dB, delayed by a phase of 0°, and filtered. Although the phase is advanced in a signal having a frequency sufficiently lower than the first frequency, it is decayed at a ratio greater than 6 dB according to the operation of the HPF. Although the phase is delayed in a signal having a frequency sufficiently higher than the first frequency, it is decayed at a ratio greater than 6 dB according to the operation of the LPF. That is, because the BPF filters the biological signal having the first frequency substantially in a delay of 0 sec and a signal of a frequency component in which a phase lead or lag (Lag time=Phase lag [°]×Frequency [Hz]) occurs is decayed at a high ratio, the effect of distortion of a waveform due to the phase lag can be substantially ignored.

### [Filtering Operation]

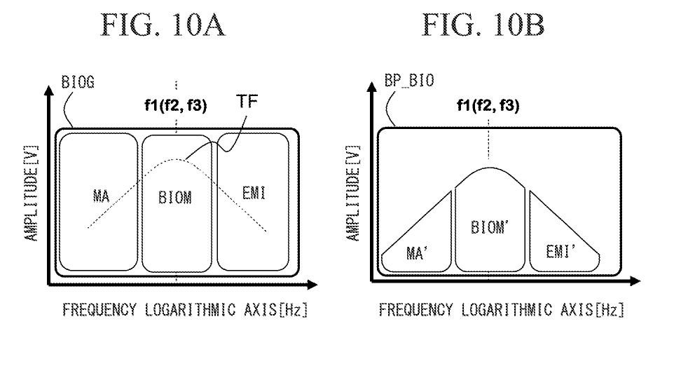

Hereinafter, the filtering operation of the BPF disclosed in this embodiment will be described using FIGS. 10A and 10B.

FIG. 10A is a graph representing amplitude-frequency characteristics of each signal constituting a biological signal BIOG wherein the vertical axis represents amplitude and the horizontal axis represents frequency. FIG. 10B is a graph illustrating amplitude-frequency characteristics of each signal constituting a filtered biological signal BP_BIO, wherein the vertical axis represents amplitude and the horizontal axis represents frequency.

First, the biological signal BIOG will be described using FIG. 10A. The biological signal BIOG includes a motion artifact MA, a biological event signal BIOM, and extraneous noise (electromagnetic interference (EMI)). The motion artifact MA is a signal of a low frequency generated when a contact state between the tissue ORG and the lead LEAD is varied according to activity of the biological body BDY. The biological event signal BIOM is a signal generated according to activity of the tissue ORG, and serves as a detection target of the peak location PK_LOCATION to be detected by the peak detecting means PK_DETECT. The biological event signal BIOM includes at least the first frequency f1. The extraneous noise EMI is a signal coming from the outside of the biological body BDA; and includes noise or the like generated from a commercial power supply or a portable telephone. In addition, the wavy line illustrated in FIG. 10A is a transfer function (TF) of the BPF.

The filtered biological signal BP_BIO is represented by a product of the biological signal BIOG and the TF of the BPF as illustrated in FIG. 10B. According to a filtering operation of the BPF, amplitudes of a motion artifact MA' and an extraneous noise EMI' are suppressed compared to a biological event signal BIOM'. According to the filtering operation, it is possible to detect a peak location with high precision in the peak detecting means PK_LOCATION connected to a subsequent stage of the BPF.

As described above, because the pole frequencies of the HPF and LPF are closely located each other in the BPF disclosed in this embodiment, it is possible to substantially cancel the signal delay at the center frequency and filter a biological event signal BIOM including the first frequency f1 necessary to detect the peak location of the biological signal from the biological signal BIOG in a delay time less than that of the related art.

### Second Embodiment

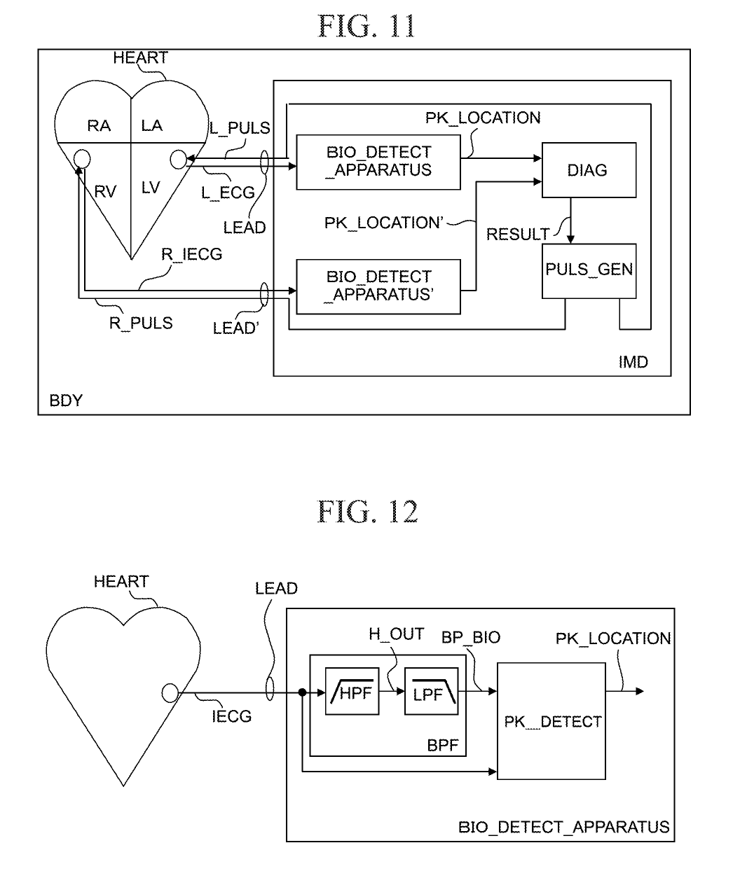

Hereinafter, an IMD according to the second embodiment will be described using FIG. 11. FIG. 11 is a diagram illustrating a relationship between a configuration of the IMD according to each embodiment of the present invention and a heart HEART to be treated by the IMD.

First, the biological body BDY will be described. The biological body BDY has the heart HEART and the IMD is implanted under the skin. The heart HEART includes a right atrium (RA), a left atrium (LA), a right ventricle (RV), and a left ventricle (LV).

Next, the IMD will be described. The IMD includes a biological signal detecting apparatus BIO_DETECT_APPARATUS, a biological signal detecting apparatus BIO_DETECT_APPARATUS', a diagnosis means DIAG and a therapy signal generating means PULS_GEN.

The biological signal detecting apparatus BIO_{—} DETECT_{—} APPARATUS is connected to the LV via the lead LEAD, and also connected to the diagnosis means DIAG.

The biological signal detecting apparatus BIO_DETECT_APPARATUS' is connected to the RV via the lead LEAD', and also connected to the diagnosis means DIAG.

The diagnosis means DIAG is connected to the biological signal detecting apparatuses BIO_DETECT_APPARATUS and BIO_{—} DETECT_{—} APPARATUS' and the therapy signal generating means PULS_GEN.

The therapy signal generating means PULS_GEN is connected to the diagnosis means DIAG and also connected to the LV via the lead LEAD and the RV via the lead LEAD'.

An IECG signal L_IECG from the LV is input to the biological signal detecting apparatus BIO_DETECT_APPARATUS via the lead LEAD. The biological signal detecting apparatus BIO_DETECT_APPARATUS detects a location serving as approximately a vertex of the R wave of the IECG signal L_IECG, and outputs a peak location PK_LOCATION to the diagnosis means DIAG.

An IECG signal R_IECG from the RV is input to the biological signal detecting apparatus BIO_DETECT_APPARATUS' via the lead LEAD'. The biological signal detecting apparatus BIO_DETECT_APPARATUS' detects a location serving as approximately a vertex of the R wave of the IECG signal R_IECG, and outputs a peak location PK_LOCATION' to the diagnosis means DIAG.

The peak location PK_LOCATION from the biological signal detecting apparatus BIO_DETECT_APPARATUS is input to the diagnosis means DIAG, and the peak location PK_LOCATION' from the biological signal detecting apparatus BIO_DETECT_APPARATUS' is input to the diagnosis means DIAG. Based on information of the peak location PK_LOCATION and the peak location PK_LOCATION', the diagnosis means DIAG outputs a diagnosis result RESULT representing a heart state to the therapy signal generating means PULS_GEN. Contents of the diagnosis result RESULT are the NSR, the VT, the VF, asynchrony of both right ventricles, and the like.

In the diagnosis means DIAG, a counted heart rate of up to 40 bpm is diagnosed as bradycardia, and a counted heart rate of 41 to 145 bpm is diagnosed as NSR. In addition, the diagnosis means DIAG diagnoses that ventricular contraction is asynchronous when the peak location PK_LOCATION' does not appear within 10 ms after appearance of the peak location PK_LOCATION or when the peak location PK_LOCATION does not appear within 10 ms after appearance of the peak location PK_LOCATION'.

The diagnosis result RESULT is input from the diagnosis means DIAG to the therapy signal generating means PULS_GEN, and therapy signals R_PULS and L_PULS corresponding to the diagnosis result RESULT are output to the RV and the LV, respectively. The therapy signals R_PULS and L_PULS are not generated when the diagnosis result RESULT is the NRS, but anti-tachycardia pacing is performed on the ventricle in which the VT has occurred when the diagnosis result is the VT. When the diagnosis result RESULT is the VF, powerful electric shocks are applied for defibrillation therapy for the ventricle in which the VT has occurred. When non-synchronization is observed in the ventricular contraction, the ventricle of the side at which the appearance of a peak location PK_LOCATION′ or PK_LOCATION″ has been delayed is paced.

### [Biological Signal Detecting Apparatus]

Hereinafter, the biological signal detecting apparatus BIO_DETECT_APPARATUS according to the second embodiment of the present invention will be described with reference to FIG. 12. Because internal configurations and operations of the biological signal detecting apparatuses BIO_DETECT_APPARATUS and BIO_DETECT_APPARATUS' are the same, only the biological signal detecting apparatus BIO_DETECT_APPARATUS will be described. In addition, because the same operation and effect are obtained when the installation position of the lead is a ventricle or an atrium other than the LV, the lead will be described later as being connected to the heart HEART. A signal generated by the heart HEART will be described later as an IECG signal IECG which does not limit a position of the ventricle or atrium.

FIG. 12 is a diagram illustrating a configuration of the biological signal detecting apparatus BIO_DETECT_APPARATUS according to this embodiment.

### [Configuration]

The biological signal detecting apparatus BIO_DETECT_APPARATUS includes the BPF and a peak detecting means PK_DETECT.

The BPF includes an HPF and an LPF.

The biological signal detecting apparatus BIO_DETECT_APPARATUS is connected to a heart HEART via a lead LEAD. The HPF is connected to the lead LEAD and the LPF. The LPF is connected to the HPF and the peak detecting means PK_DETECT. The peak detecting means PK_DETECT is connected to the lead LEAD and the LPF.

An IECG signal IECG from the heart HEART is input to the biological signal detecting apparatus BIO_DETECT_APPARATUS via the lead LEAD.

An IECG signal is input to the HPF as a biological signal BIOG including an R wave (biological event signal BIOM) of 12 Hz as a first frequency f1, and output as a high-pass output H_OUT by decaying a frequency component less than or equal to a second frequency f2 included in the input IECG signal IECG.

The high-pass output H_OUT is input to the LPF, and output as a filtered biological signal BP_BIO by decaying a frequency component greater than or equal to a third frequency f3 within the input high-pass output H_OUT.

However, in this embodiment, the second frequency f2 is 10 Hz, and the third frequency f3 is 15 Hz.

The IECG signal IECG and the filtered biological signal BP_BIO are input to the peak detecting means PK_DETECT. The peak detecting means PK_DETECT detects an R wave with high precision by processing the filtered biological signal BP_BIO obtained after suppressing a T wave (a signal belonging to a frequency less than 10 Hz) or EMI (a signal belonging to a frequency higher than 15 Hz), detects an accurate location of the R wave by matching the detected R wave against the IECG signal IECG, and outputs the detected location as a peak location PK_LOCATION. A detailed algorithm of the peak detecting means PK_DETECT will be described later.

### [HPF]

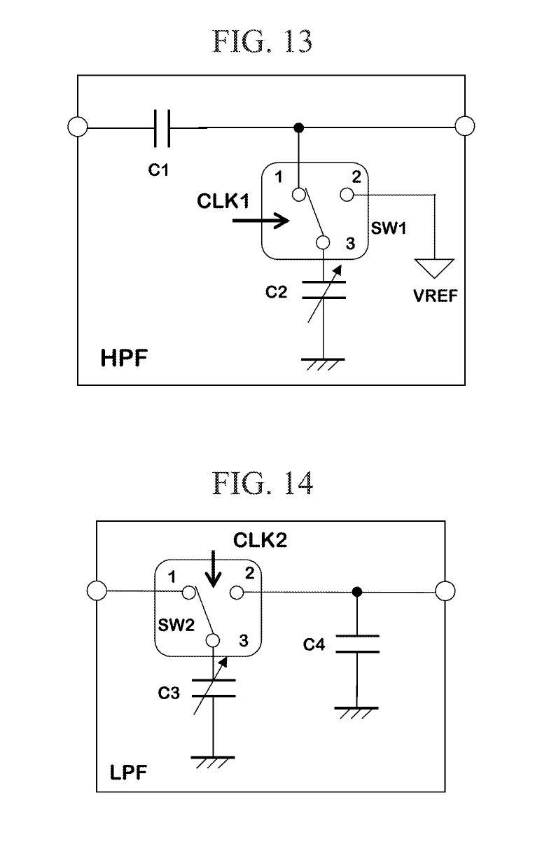

Hereinafter, the HPF will be described in further detail using FIG. 13.

### [Configuration]

The HPF includes a capacitor C1, a switch SW1, and a variable capacitor C2.

A first terminal of the capacitor C1 is connected to an input terminal of the HPF, and a second terminal is connected to an output terminal of the HPF and a first terminal of the switch SW1. The first terminal of the switch SW1 is connected to the second terminal of the capacitor C1 and the output terminal of the HPF. A second terminal of the switch SW1 is connected to a reference voltage VREF. A third terminal of the switch SW1 is connected to a first terminal of the variable capacitor C2.

The first terminal of the variable capacitor C2 is connected to the third terminal of the switch SW1, and a second terminal of the variable capacitor C2 is connected to a ground GND. A clock CLK1 in which a high (H) level and a low (L) level are cyclically varied at a frequency f_{CLK1} is input from an input terminal (not illustrated) to the switch SW1.

### [Operation]

First, an equivalent resistor including the switch SW1 and the variable capacitor C2 will be described. In the switch SW1, first and third terminals are connected when the clock CLK1 is at the H level, and second and third terminals are connected when the clock CLK1 is at the L level. A circuit including the switch SW1 that performs such an operation and the variable capacitor C2 is equivalent to a resistor having a resistance value of Reff1 in which a first terminal is connected to the second terminal of the capacitor C1 and the output terminal of the HPF and a second terminal is connected to a reference voltage VREF. Because a detailed operation principle is disclosed in “Analog Filter Design” translated by Yanagisawa Ken, ISBN 4-87184-041-7, pp. 554, it is omitted here.

Reff1=1/(C₂·f_{CLK1}) (1)

The HPF having such a configuration is referred to as a first order HPF, and the second frequency f2 set as a cutoff frequency is given as shown in the following Expression (2).

f2=1/(2π·Reff1·C₁)=(C₂·f_{CLK1})/(2π·C₁) (2)

### [LPF]

Hereinafter, the LPF will be described in detail using FIG. 14.

### [Configuration]

The LPF includes a variable capacitor C3, a switch SW2, and a capacitor C4.

A first terminal of the variable capacitor C3 is connected to a third terminal of the switch SW2, and a second terminal of the variable capacitor C3 is connected to a ground GND.

A first terminal of the switch SW2 is connected to an input terminal of the LPF, and a second terminal of the switch SW2 is connected to an output terminal of the LPF and a first terminal of the capacitor C4.

The first terminal of the capacitor C4 is connected to the second terminal of the switch SW2 and connected to the output terminal of the LPF, and the second terminal of the capacitor C4 is connected to the ground GND.

A clock CLK2 in which the H level and the L level are cyclically varied at a frequency f_{CLK2} is input from an input terminal (not illustrated) to the switch SW2.

### [Operation]

First, an equivalent resistor including the switch SW2 and the variable capacitor C3 will be described. In the switch SW2, first and third terminals are connected when the clock CLK2 is at the H level, and second and third terminals are connected when the clock CLK2 is at the L level. A circuit including the switch SW2 that performs such an operation and the variable capacitor C2 is equivalent to a resistor having a resistance value of Reff2 in which a first terminal is connected to an input terminal of the LPF and a second terminal is connected to a first terminal of the capacitor C4 and an output terminal of the LPF.

Reff2=1/(C₃·f_{CLK2}) (3)

The LPF having such a configuration is referred to as a first order LPF, and the third frequency f3 set as a cutoff frequency is given as shown in the following Expression (4).

f3=1/(2π·Reff119 C₄)=(C₂·f_{CLK2})/(2πC₃) (4)

### [BPF]

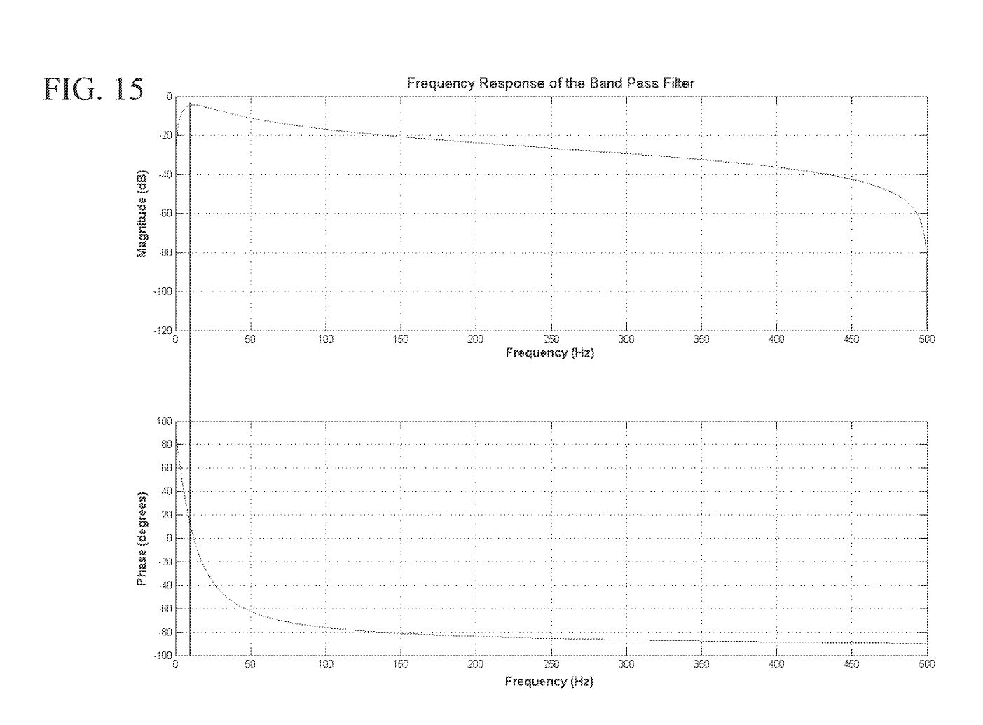

Hereinafter, a phase lag occurring in the BPF will be described using FIG. 15. Because the center frequency of the BPF, the cutoff frequency of the HPF, and the cutoff frequency of the LPF described in the first embodiment are all the same, a phase lag occurring in the BPF is 0.

The cutoff frequency of the HPF in the second embodiment is set to 10 Hz and the cutoff frequency of the LPF is set to 15 Hz. Bode diagrams obtained by simulating characteristics of such filters are illustrated in FIG. 15.

The upper graph of FIG. 15 represents gain-frequency characteristics of the BPF, wherein the horizontal axis represents frequency (linear display) and the vertical axis represents gain. The graph on the bottom of FIG. 15 represents frequency-phase characteristics of each filter, wherein the horizontal axis represents frequency (linear display) and the vertical axis represents a phase lead (a positive is a phase lead and a negative is a phase lag).

As can be seen from simulation results, because the phase lag at the center frequency (12.5 Hz) is also about 0° in this embodiment, the R wave having a frequency component of 12.5 Hz is not substantially delayed and reaches the peak detecting means PK_DETECT.

A signal of the T wave or the like at a frequency sufficiently lower than 10 Hz is output as part of the high-pass output H_OUT (in a waveform obtained by differentiating the T wave) at a time earlier than that the actual T wave. However, because the T wave is sufficiently suppressed according to the operation of the HPF at a frequency sufficiently lower than 10 Hz, the amplitude of the T wave capable of being observed as the output of the HPF is small and ignorable compared to the R wave.

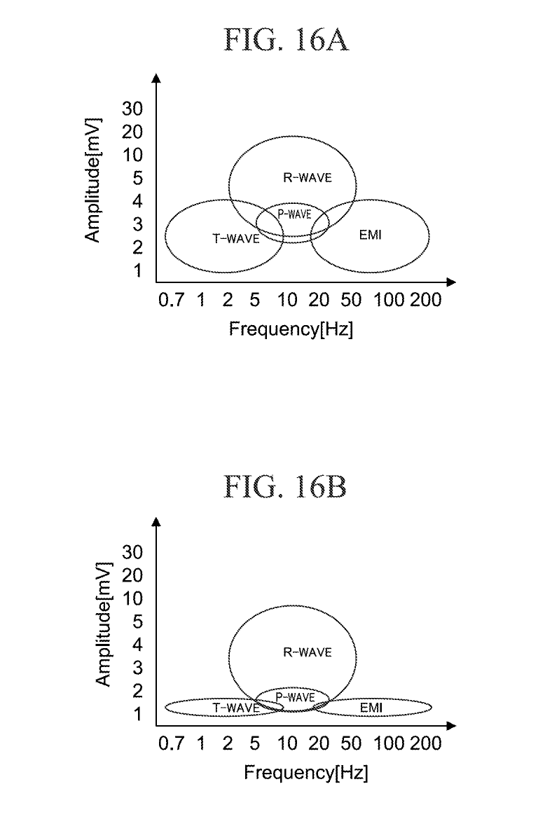

A signal of the EMI or the like at a frequency sufficiently higher than 15 Hz is output as part of a filtered IECG signal BP_IECG (in a waveform obtained by integrating the EMI) at a time later than that the actual EMI. However, because the EMI is sufficiently suppressed according to the operation of the LPF at a frequency sufficiently higher than 15 Hz, the amplitude of the EMI capable of being observed as the output of the LPF is small and ignorable compared to the R wave. A graph representing this effect is illustrated in FIGS. 16A and 16B. FIG. 16A is a diagram illustrating relationships of frequencies and amplitudes of T, R, and P waves and EMI constituting an IECG signal IECG created based on K. A. Ellenbogen, G. N. Kay C-P. Lau, and B. L. Wilkoff, “Clinical Cardiac Pacing, Defibrillation, and Resynchronization Therapy,” Elsevier, 3rd edition, 2007, ISBN 1-4160-2536-7.

In addition, FIG. 16B is a diagram illustrating a relationship between a frequency and amplitude of each signal constituting the filtered biological signal BP_BIO obtained according to a filtering operation of the BPF. As can be seen by comparing FIGS. 16A and 16B, the BPF can accurately perform peak detection of the R wave by the subsequent-stage peak detecting means PK_DETECT by suppressing the T wave and EMI to a sufficiently small amplitude.

Because the BPF disclosed in this embodiment sets cutoff frequencies of the first order HPF having the cutoff frequency at the second frequency and the first order LPF having the cutoff frequency at the third frequency to frequencies ((Center Frequency −2.5 Hz) and (Center Frequency +2.5 Hz)) close to the center frequency as described above, the phase lead around the second frequency generated by the HPF and the phase lag around the third frequency generated by the LPF are offset and therefore the delay of a signal occurring at the center frequency can be substantially canceled.

In addition, by setting the HPF to a switched capacitor type in which an error of the cutoff frequency is mainly determined by relative manufacturing errors of C₁ and C₂ and setting the LPF to a switched capacitor type in which an error of the cutoff frequency is mainly determined by relative manufacturing errors of C₃ and C₄, it is possible to set the cutoff frequency of the BPF with high precision of about several percent without performing correction such as trimming. Because the BPF can be executed in a simple configuration (a small number of elements) by setting the orders of the HPF and the LPF to the first order, it is possible to reduce an area of a semiconductor chip necessary to mount the BPF.

### [Peak Detecting Means]

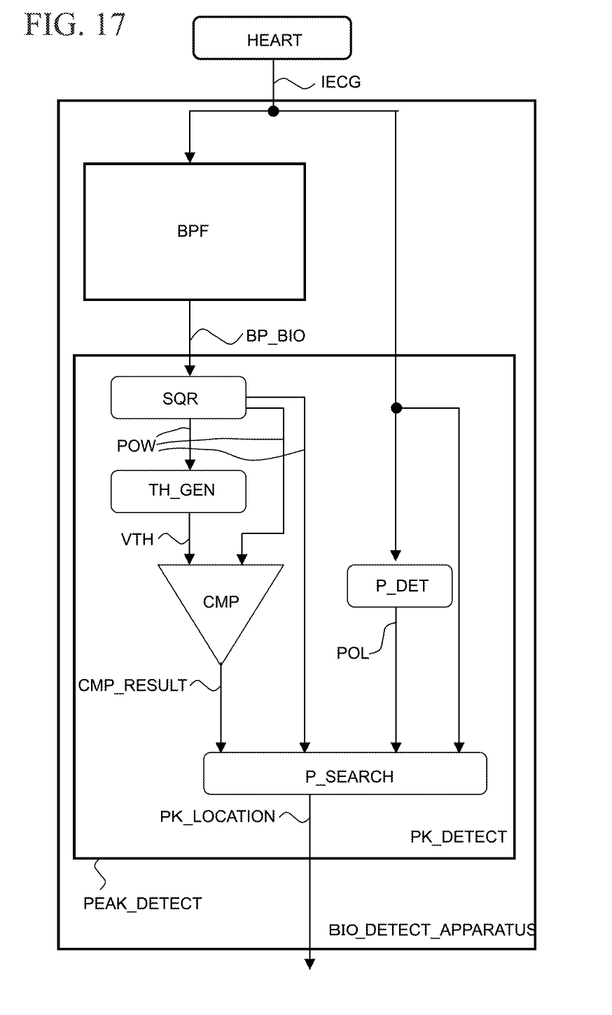

Hereinafter, details of the peak detecting means PK_DETECT disclosed in this second embodiment will be described using FIG. 17. For description of an operation principle, the heart HEART and the BPF not included in the peak detecting means PK_DETECT will also be disclosed.

### [Configuration]

The peak detecting means PK_DETECT can be expressed as a functional block within a microcontroller. The peak detecting means PK_DETECT holds a result in a memory of microcontroller which is obtained by performing analog to digital (A/D) conversion on an output of the BPF. The processing function will be described later. The peak detecting means PK_DETECT includes a square calculation means SQR, a threshold generating means TH_GEN, a comparison means CMP, a polarity determination means P_DET, and a peak search means P_SEARCH.

A filtered biological signal BP_BIO is input from the BPF to the square calculation means SQR, and IECG power is output to the threshold generating means TH_GEN, the comparison means CMP, and the peak search means P_SEARCH.

The IECG power POW is input from the square calculation means SQR to the threshold generating means TH_GEN, and a threshold voltage VTH is output to the comparison means CMP.

The threshold voltage VTH from the threshold generating means TH_GEN and the ECG power POW from the square calculation means SQR are input to the comparison means CMP, and the comparison result CMP RESULT is output to the peak search means P_SEARCH. The IECG signal IECG from the heart HEART is input to the polarity determination means P_DETECT, and a polarity determination result POL is output to the peak search means P_SEARCH.

The IECG signal IECG from the heart, the IECG power POW from the square calculation means SQR, the comparison result CMP RESULT from the comparison means CMP, and the polarity determination result POL from the polarity determination means P_DET are input to the peak search means P_SEARCH, and the R-wave location R_LOCATION is output. The operation of each block will be described hereinafter.

### [Operation]

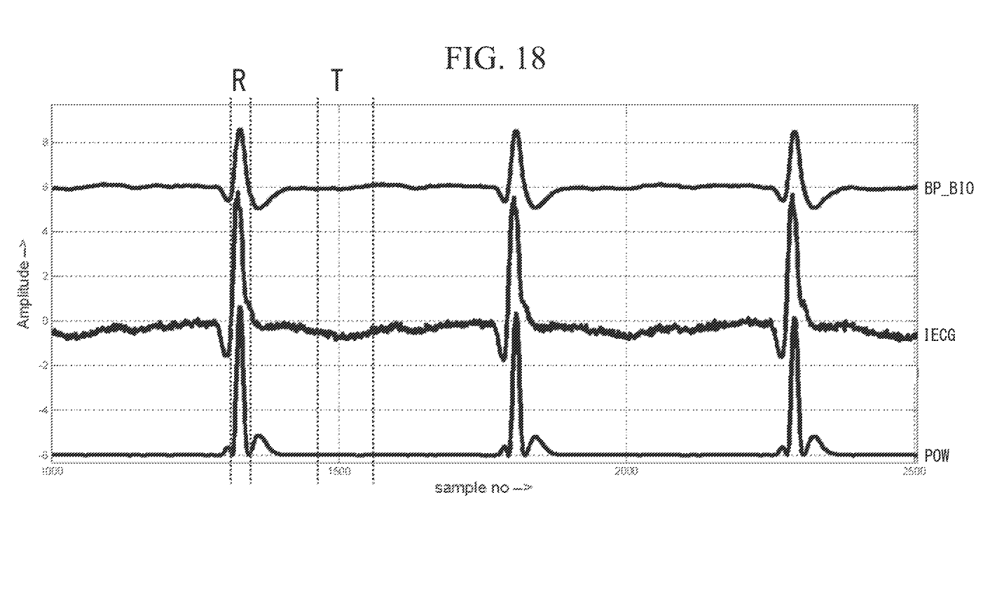

Hereafter, operations of the heart HEART, the BPF, and the square calculation means SQR will be described using FIG. 18. The horizontal axis of FIG. 18 represents time [sec]. The vertical axis of the IECG signal and the filtered biological signal BP BIO represents voltage [V], and the vertical axis of the IECG power represents power [V²].

The heart HEART generates the IECG signal as illustrated on the middle of FIG. 18. The IECG signal IECG is input to the BPF, the polarity determination means P_DET, and the peak search means PK_SEARCH.

The BPF is a block which performs an operation of suppressing a signal having a frequency component such as a T wave or EMI, and details thereof have already been described. The BPF filters the IECG signal IECG and outputs the filtered biological signal BP_BIO to the square calculation means SQR.

A waveform of the filtered biological signal BP_BIO is illustrated on the top of FIG. 16A. In the filtered biological signal BP_BIO, the amplitude of undulation occurring at the timing of the T wave and high-frequency EMI superimposed on the IECG signal IECG are suppressed.

The square calculation means SQR outputs a result obtained by squaring the input filtered biological signal BP_BIO as the IECG power POW. A waveform of the IECG power POW is illustrated on the bottom in FIG. 18. An absolute value of the filtered biological signal BP BIO is calculated by squaring the filtered biological signal BP_BIO, and the R wave of a patient whose polarity of the IECG signal IECG is reversed (the vertex of the R wave appears in a negative direction) due to the effect of an installation state of the lead LEAD can be handled regardless of polarity. In addition, it is possible to implement the enlargement (S/N improvement) of an amplitude difference of the R wave for the T wave or EMI by squaring the filtered biological signal BP_BIO. When the amplitude of the R wave in the filtered biological signal BP_BIO is 2 mV and the amplitude of the T wave is 0.2 mV, the amplitude ratio is 10:1. On the other hand, the amplitude (power) of the R wave after square calculation is 4 mV², the amplitude of the T wave is 0.04 mV², and the amplitude (power) ratio is 100:1.

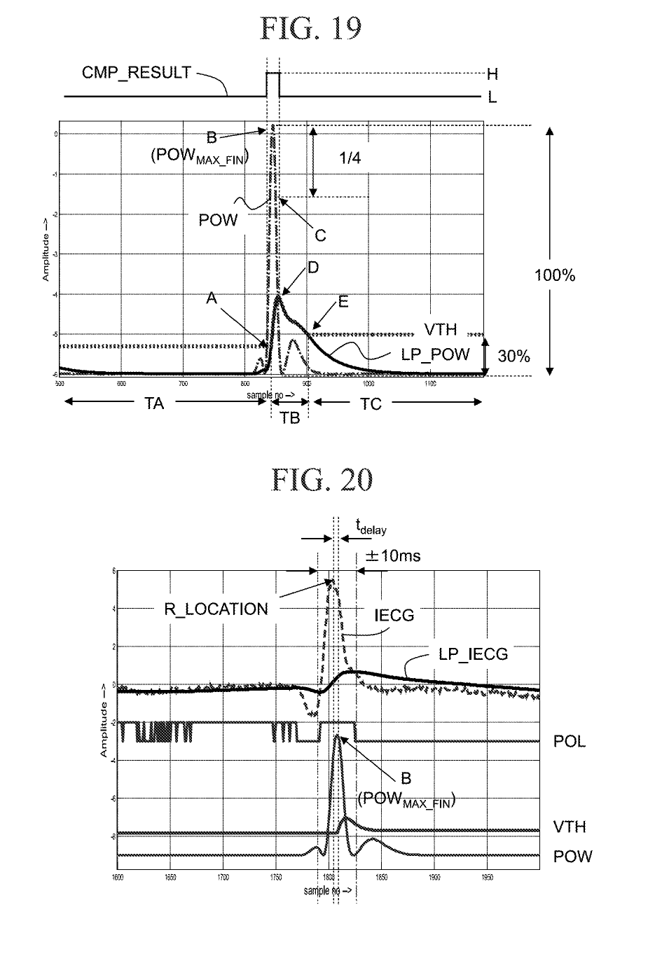

Hereinafter, operations of the threshold generating means TH_GEN and the comparison means CMP will be described using FIG. 19. The horizontal axis of FIG. 19 represents time [sec]. The vertical axis of the comparison result CMP_RESULT represents a logic level. Each of vertical axes of low-pass power LP_POW, a threshold voltage VTH, and IECG power POW represents power [V²].

The threshold generating means TH_GEN generates and outputs the threshold voltage VTH using the input ECG power POW. The threshold generating means TH_GEN generates a result obtained by multiplying a signal after low-pass filtering in an LPF of 4 Hz by 1.2 as low-pass power LP_POW. This signal is illustrated in FIG. 19. The threshold generating means TH_GEN compares 30% of a maximum value of the IECG power POW generated by the immediately preceding R wave to a value of the low-pass power LP_POW, and outputs higher power as the threshold voltage VTH. Because 30% of the maximum value (POW_{MAS}_{_{—}}_{FIN}) of the IECG power POW generated by the immediately preceding R wave is greater than a value of the low-pass power LP_POW in intervals TA and TC of FIG. 19, a value of the threshold voltage VTH is fixed to 30% of the maximum value of the IECG power POW generated by an immediately preceding R wave. In the interval TB, because 30% of the maximum value of the IECG power POW generated by the immediately preceding R wave is less than the value of the low-pass power LP_POW, it is consistent with the value of the low-pass power LP_POW.

The comparison means CMP is a block which outputs a result obtained by comparing the input IECG power POW to the threshold voltage VTH for the magnitude relation as a comparison result CMP_RESULT. When the IECG power POW is greater than or equal to the threshold voltage VTH, the comparison result CMP_RESULT is at the “H” level. When the IECG power POW is less than the threshold voltage VTH, the comparison result CMP_RESULT is at the “L” level. The waveform of the comparison result CMP_RESULT is illustrated on the top in FIG. 19.

Hereinafter, operations of the polarity determination means P_DET and the peak search means P SEARCH will be described using FIGS. 19 and 20. The horizontal axis of FIG. 20 represents time [sec]. The vertical axis of the polarity determination result POL represents a logic level. Each of the vertical axes of the IECG signal IECG and the low-pass IECG signal LP_IECG represents voltage [V], and the vertical axis of the IECG power POW represents power [V²].

The polarity determination means P_DET determines the polarity of a peak location of the IECG signal IECG using the input IECG signal IECG, and outputs the determined polarity as the polarity determination result POL. As illustrated in FIG. 20, the polarity determination means P_DET generates a low-pass IECG signal LP_IECG by performing low-pass filtering on the IECG signal IECG in a low-pass filter in which a cutoff frequency (not illustrated) is 2 Hz, and compares the generated low-pass IECG signal LP_IECG to the IECG signal for magnitude comparison. When the IECG signal IECG is greater than or equal to the low-pass IECG signal LP_IECG, the polarity determination result POL is at the “H” level. When the IECG signal IECG is less than the low-pass IECG signal LP_IECG, the polarity determination result POL is at the “L” level. When the polarity determination result POL is at the “H” level, this means that a determination result of the polarity of the IECG signal IECG is positive. When the polarity determination result POL is at the “L” level, this means that the determination result of the polarity of the IECG signal IECG is negative.

The peak search means P_SEARCH specifies a location of the R wave using the input IECG signal IECG the IECG power POW, the comparison result CMP_RESULT, and the polarity determination result POL, and outputs the specified location as the R-wave location R_LOCATION.

As illustrated in FIG. 19, the peak search means P_SEARCH starts a comparison with a value of IECG power held in the memory within the microprocessor when the comparison result CMP_RESULT is switched to the “H” level. When the IECG power value POW [k] at a time t [k] is greater than maximum power POW_{MAX} at a time t [k-1], POW [k] becomes new POW_{MAX}. This maximum value search ends when the condition of the following Expression (5) has been satisfied.

(¾)·POW_{MAX}>POW[k] (5)

In FIG. 19, when the maximum value search is started at a point A, a value of POW_{MAX} is continuously updated up to a point B. Although the value of POW_{MAX} is not updated after the point B, the maximum value search continues. Because Expression (5) is established after a point C, the maximum value search ends and the final value and timing of POW_{MAX} are fixed.

If the final value and timing of POW_{MAX} are fixed, the peak search means P_SEARCH detects a maximum value of the IECG signal IECG after A/D conversion within previous and subsequent 10 ms using the timing (the point B of FIG. 19 or 20) at which the final value of POW_{MAX} is maximized as the origin, and outputs its timing as the R-wave location R_LOCATION (FIG. 20).

### [Simulation Results]

In order to observe the above-described effect of the present invention, signal processing executed by a biological signal detecting apparatus PK_DETECT_APPARATUS disclosed in the second embodiment was implemented in program codes of Matlab and simulation experiments were performed. Different NSRs of 5 patients were extracted from an IECG waveform data collection (http://www.electrogram.com/about.html) of Ann Abor instead of IECG signals from patients and simulated by Matlab.

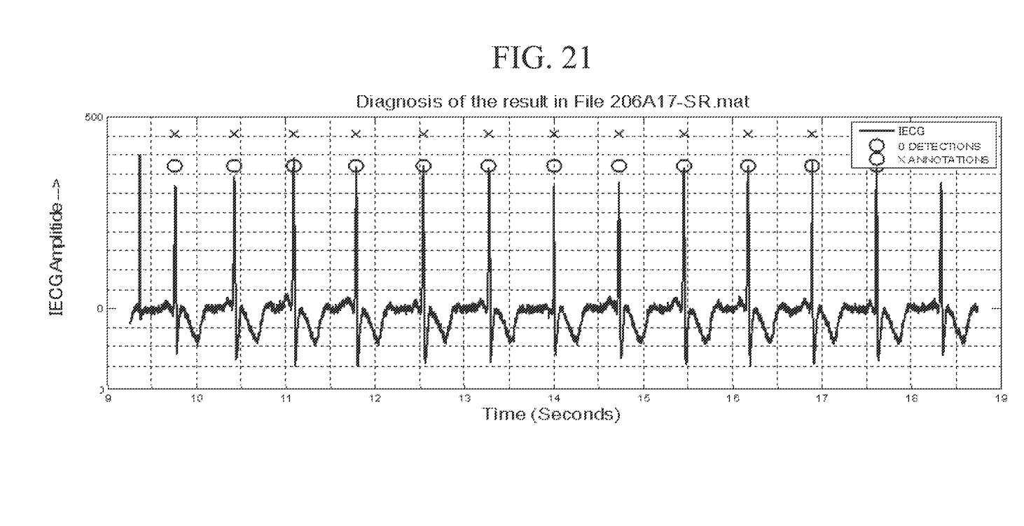

FIG. 21 illustrates simulation results representing a state of peak location detection when a representative NSR waveform was input to the biological signal detecting apparatus PK_DETECT_APPARATUS, wherein the vertical axis represents amplitude, the horizontal axis represents time, a mark 0 represents a detected location of the R wave, a mark X represents a location of the R wave marked by a doctor, and a solid line represents an IECG signal IECG. As can be seen from FIG. 21, it is possible to confirm that the biological signal detecting apparatus PK_DETECT_APPARATUS has detected an accurate location of the R wave.

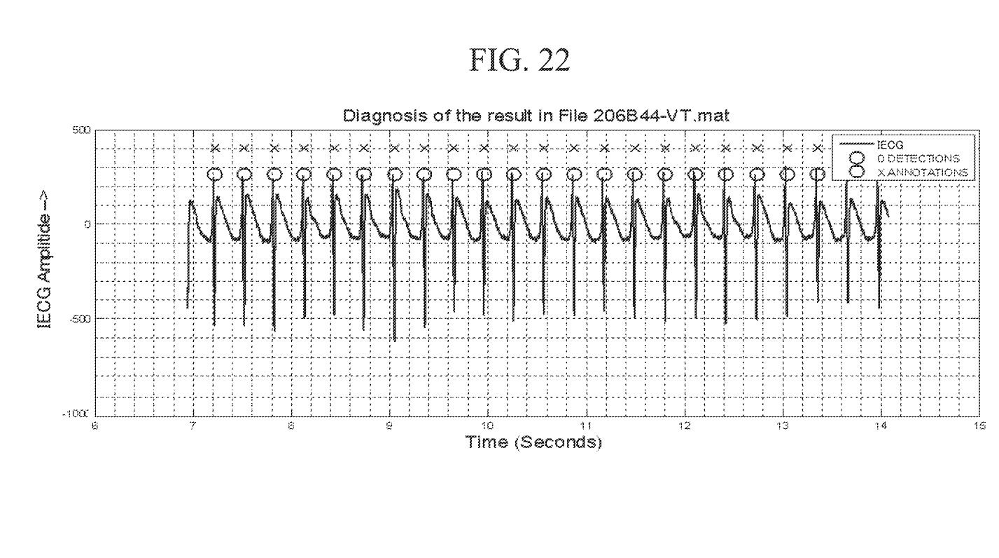

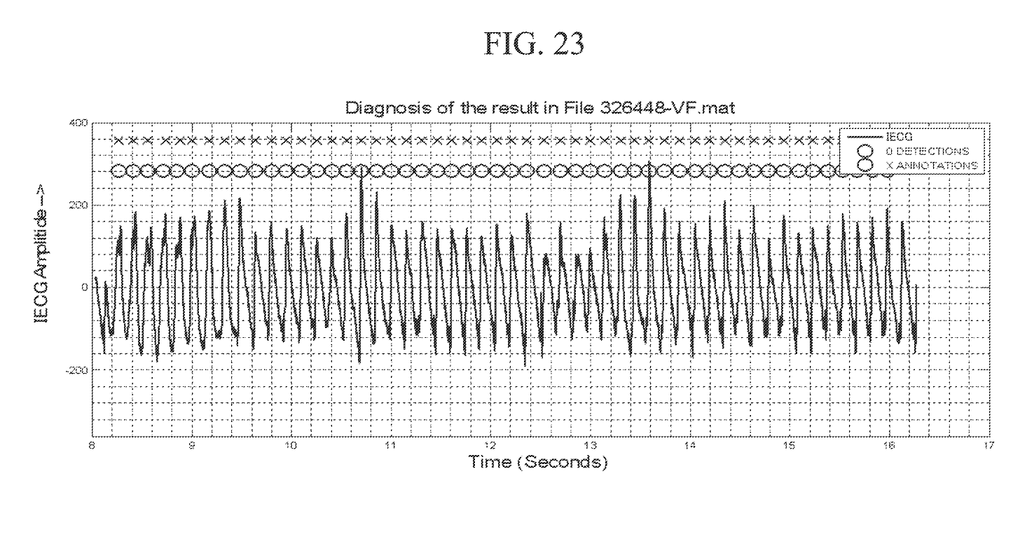

Likewise, as confirmed, it is possible to extract different VT waveforms of 10 patients and different VF waveforms of 4 patients and accurately diagnose a VT or VF from each waveform. A state of R-wave detection in a representative VT waveform is illustrated in FIG. 22, and a state of R-wave detection in a representative VF waveform is illustrated in FIG. 23.

Different NSR waveforms of 5 patients were extracted and a result obtained by generating a histogram from a location of the P wave marked by the doctor and a deviation amount (t_{delay} of FIG. 20) of the timing of POW_{MAX}_{_{—}}_{FIN} is illustrated in FIG. 24. The horizontal axis of FIG. 24 represents a delay time (a negative side is a delay) and the vertical axis represents a frequency of a detected R-wave location. As can be seen from the graph, because the delay time of the timing of POW_{MAX}_{_{—}}_{FIN} based on the location of the R wave marked by the doctor was less than 10 ms, it was possible to confirm that the peak search means P_SEARCH could detect an accurate peak location of the IECG signal IECG in a delay time within 10 ms.

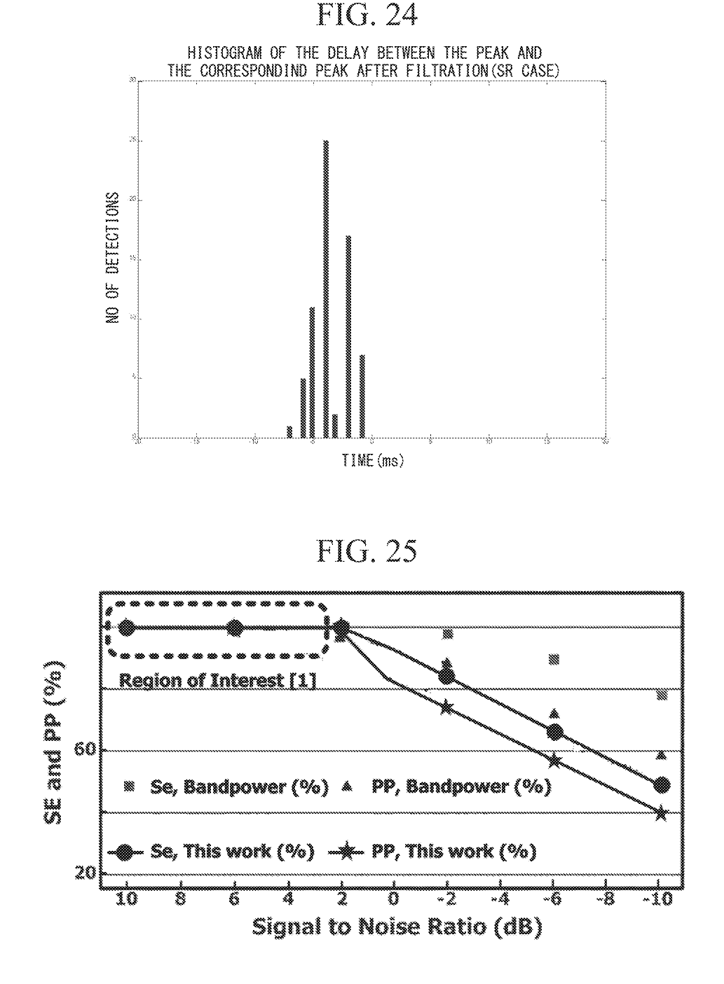

In addition, results of simulation of R-wave detection precision performed using an IECG signal obtained by intentionally superimposing a disturbance signal (noise) on the IECG signal IECG are illustrated in FIG. 25. The horizontal axis represents a ratio of S/N of noise superimposed on the IECG signal IECG (signal), and the vertical axis represents sensitivity (SE), which is a detection precision index, and positive predictivity (PP). A graph of “Se, Bandpower” among graphs represents SE of an EECG signal detecting apparatus disclosed in IEEE JOURNAL OF SOLID-STATE CIRCUITS, VOL. 46, NO. 1, JANUARY 2011, and a graph of “PP, Bandpower” represents PP of the EECG signal detecting apparatus disclosed in IEEE JOURNAL OF SOLID-STATE CIRCUITS, VOL. 46, NO. 1, JANUARY 2011. These results are reported in European Patent Publication No. 2 589 332 A1 and Torfs, T.; et. al, “Ultra Low Power Wireless ECG system with Beat Detection and Real Time Impedance Measurement,” IEEE Biomedical Circuits and Systems Conference (BioCAS), 2010 IEEE, pp.33 to 36, 3 to 5 Nov. 2010.

In addition, a graph of “Se, This work” among the graphs represents SE of the EECG signal detecting apparatus disclosed in this embodiment, and a graph of “PP, This work” represents PP of the EECG signal detecting apparatus disclosed in this embodiment.

Although SE and PP of the biological signal detecting apparatus PK DETECT APPARATUS disclosed in this embodiment are inferior to SE and PP of EECG signal disclosed in IEEE JOURNAL OF SOLID-STATE CIRCUITS, VOL. 46, NO. 1, JANUARY 2011 under a condition of an S/N ratio less than or equal to 0 dB, it was confirmed that the R wave could be detected with a precision of 100% under a testing condition of S/N required for an IMD disclosed in K. A. Ellenbogen, G. N. Kay, C-P. Lau, and B. L. Wilkoff, “Clinical Cardiac Pacing, Defibrillation, and Resynchronization Therapy,” Elsevier, 3rd edition, 2007, ISBN 1-4160-2536-7.

As described above, because the IMD disclosed in this embodiment can accurately detect the R wave in a delay time less than or equal to 10 ms under an S/N condition required for the IMD, effective CTR therapy is performed. Further, because it is also possible to detect the symptoms of a VT and VF with a high precision, therapy corresponding to the symptoms is performed.

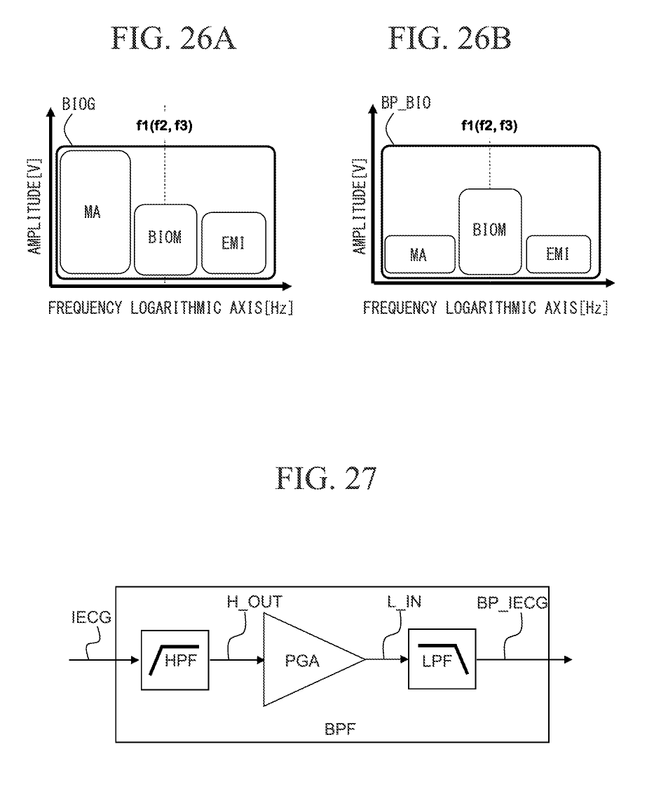

In addition, as reference, a graph for a comparison between a frequency component of a biological signal to be detected by the EECG signal detecting apparatus and a frequency component of a biological signal BIOG to be detected by an IECG signal detecting apparatus is illustrated in FIGS. 26A and 26B. The horizontal axis of each graph represents frequency and the vertical axis represents amplitude. It can be seen that FIG. 26A corresponds to a frequency component of a biological signal to be detected by the EECG signal detecting apparatus, the amplitude of a motion artifact MA is large due to variation of a contact state of an electrode attached to the skin, and extraneous noise EMI of a portable telephone, a commercial power supply, or the like coming from the outside also has amplitude which is not ignorable with respect to a biological event signal BIOM.

On the other hand, FIG. 26B corresponds to a frequency component of a biological signal to be detected by the IECG signal detecting apparatus, and the amplitude of the motion artifact MA is less than that of the EECG signal detecting apparatus due to variation of a contact state of an electrode directly attached to the heart HEART. In addition, because a biological event signal having comparatively large amplitude can be acquired from an electrode directly attached to the heart HEART, the amplitude of the biological event signal is greater than the extraneous noise EMI.

As can be seen from these graphs, since the biological signal detecting apparatus PK_DETECT_APPARATUS is optimized for the purpose of detecting an IECG signal, biological event signal BIOM can be detected with sufficiently high precision by using more simple BPF having a small delay than the filter used in the EECG signal detecting apparatus.

### [Modified Examples]

As illustrated in FIG. 27, a variable gain amplifier PGA may be inserted between the HPF and the LPF constituting the BPF. Thereby, it is possible to more accurately detect a peak location because a lost gain can also be compensated for at a center frequency of the BPF and a signal of a higher S/N ratio can be supplied to the peak detecting means PK_DETECT.

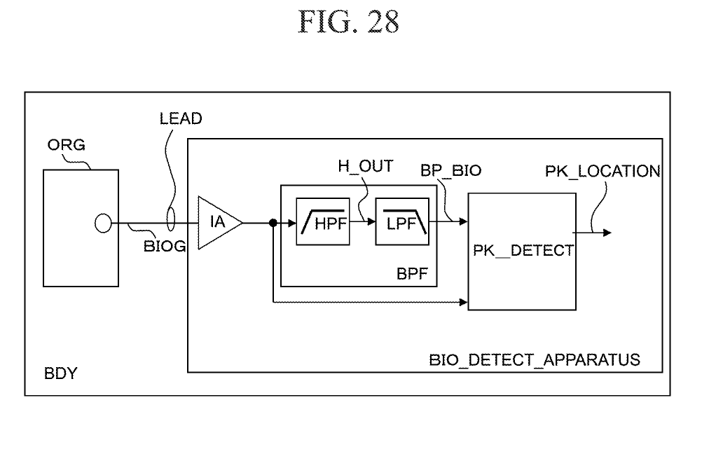

In addition, as illustrated in FIG. 28, after a signal generated from a tissue ORG is amplified by an instrumentation amplifier (IA) via the lead LEAD, the amplified signal may be input to the BPF and the peak detecting means PK_DETECT. Thereby, because in-phase noise such as EMI can be suppressed at a high ratio and a signal from which the noise is suppressed can be supplied to a subsequent-stage block, it is possible to more accurately detect a peak location.

Although only the case in which the tissue ORG is a heart HEART has been described in the embodiment example of this application, the tissue ORG may be another body organ such as a brain. When the biological signal detecting apparatus BIO_DETECT_APPARATUS is used to detect a brain wave, the present invention is effective in diagnosing epilepsy or the like.

Although an example in which the biological signal detecting apparatus is connected to both the RV and LV has been described in the second embodiment, a connection to the RV or LV may be made. In this case, although it is impossible to perform the CRT therapy, it is possible to detect the VT or VF, further detect bradycardia, and supply necessary therapy (pacing or the like) to the heart HEART. In addition, in an object for more accurately acquiring a state of the IECG signal IECG, the biological signal detecting apparatus BIO_DETECT_APPARATUS may be connected to the RA or LA via the lead.

### [Further Description]

A nonlinear phase band-pass filter results in large variations in the time delay between the output of the Compare block and the actual location of the R peak in the signal; this requires the addition of a computation-intensive Continuous Wavelet Transform (CWT) block for precise location of the R peak in the signal. Using a simple time domain search on the rough time window would yield unacceptable accuracy results in the presence of artifacts in the signal.

According to an exemplary embodiment of the present description, in the new method, the use of a linear phase band-pass filter results in the output of the compare window having a predictable time delay to the actual location of the R peak in the signal; this eliminates the need for a complex CWT operation. The Locate block can be used to estimate the location of the R peak with sufficient accuracy. A simple time domain search on a small window yields an accurate and robust R peak location, and there is no more need for a computation-intensive search function such as CWT.

The new algorithm according to an exemplary embodiment of the present invention, can be implemented with very low power consumption, since there are no computationally intensive blocks; this is important for wearable and implantable devices which need to operate for a long time from a small battery. It is clearly seen from the comparison of two algorithms that the CWT operation is eliminated, leading low-power dissipation.

The new algorithm according to an exemplary embodiment of the present invention can be implemented with very low latency, since it permits to locate the R peak in real time within a small time window, without the need to search a larger time window of the IECG signal using a method such as CWT; this is important for closed-loop applications where a stimulus needs to be activated within a short time delay after an abnormality is observed.

It is noted that the state of the art in low-power, low-latency algorithms is based on an exponential decay threshold, see previous section, which while it can achieve comparable or better power and latency than our new algorithm, suffers from a lack of robustness against artefacts (e.g. motion artefacts, large T waves, etc.), which will cause incorrectly detected R peaks.

According to one exemplary embodiment of the present description the linear phase band-pass filter may be implemented in the analogue domain. This results in a more power efficient solution than if the filtering has to be implemented in the digital domain, and which can in turn increase the battery lifetime of the device implementing this algorithm.

According to another exemplary embodiment of the present description, the algorithm parameters may be optimized for use with intracardiac signals. The low latency of the algorithm permits in this case to diagnose certain abnormalities and apply a timely treatment stimulus.

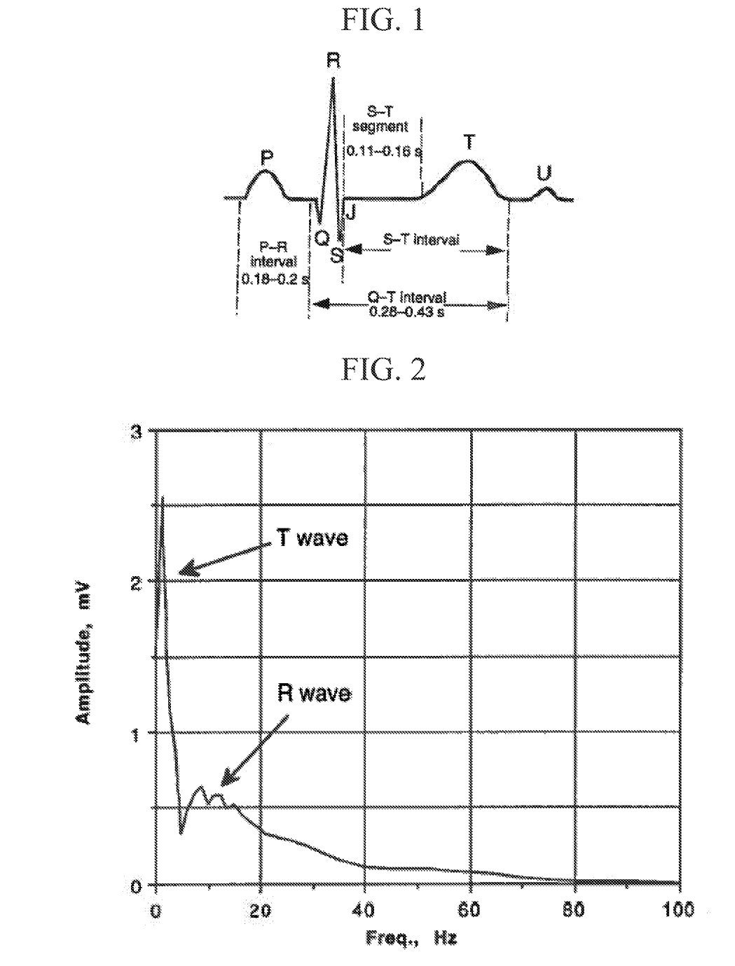

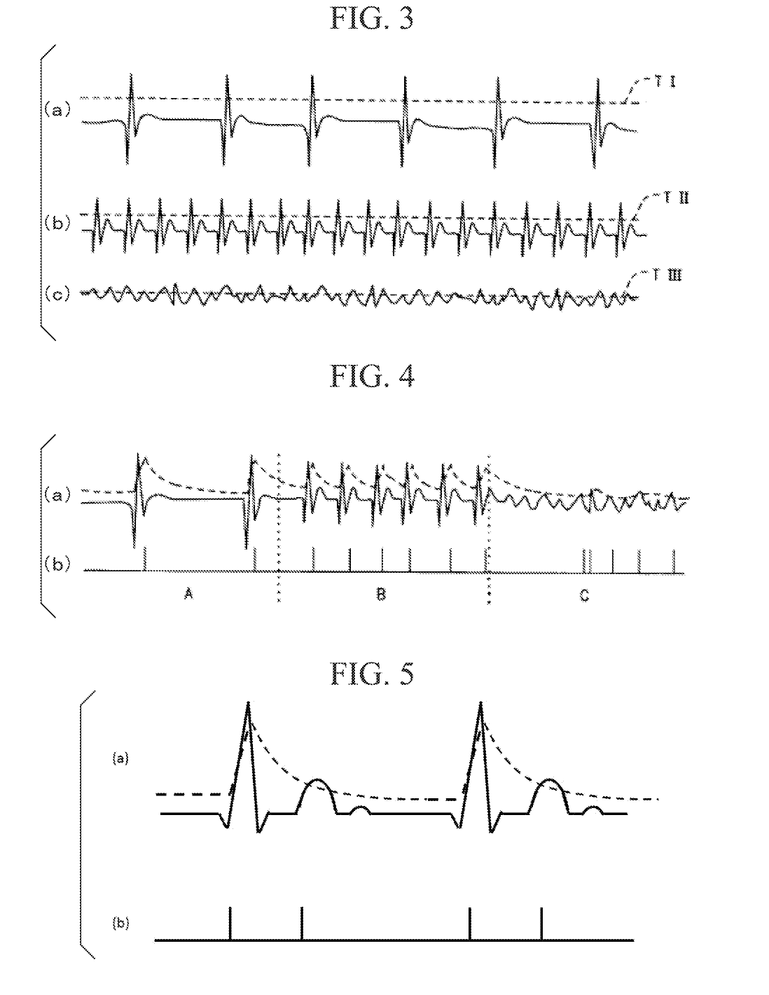

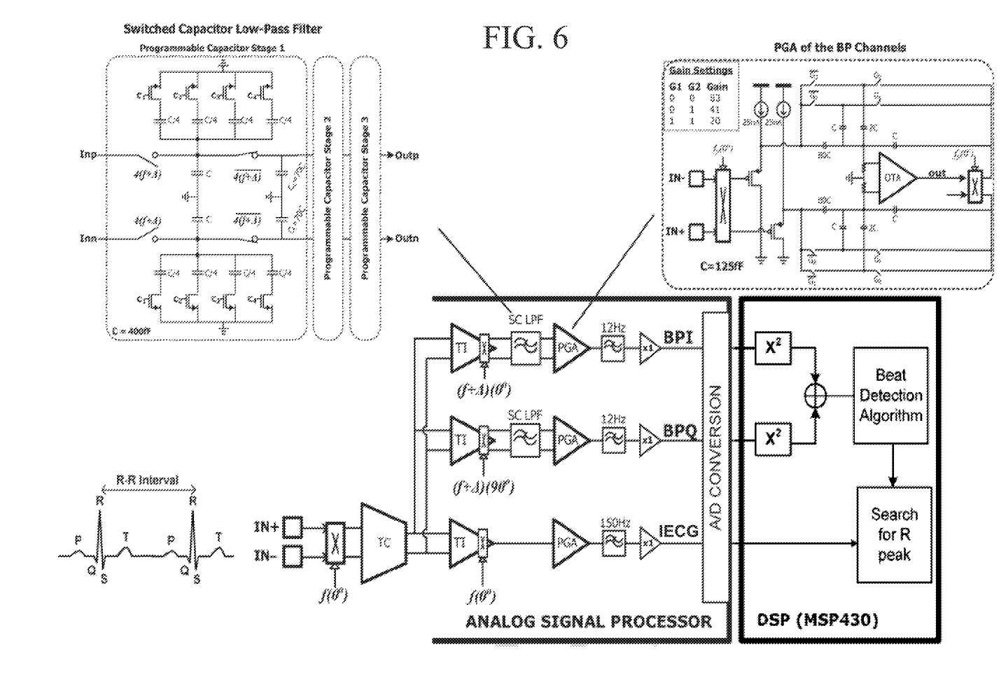

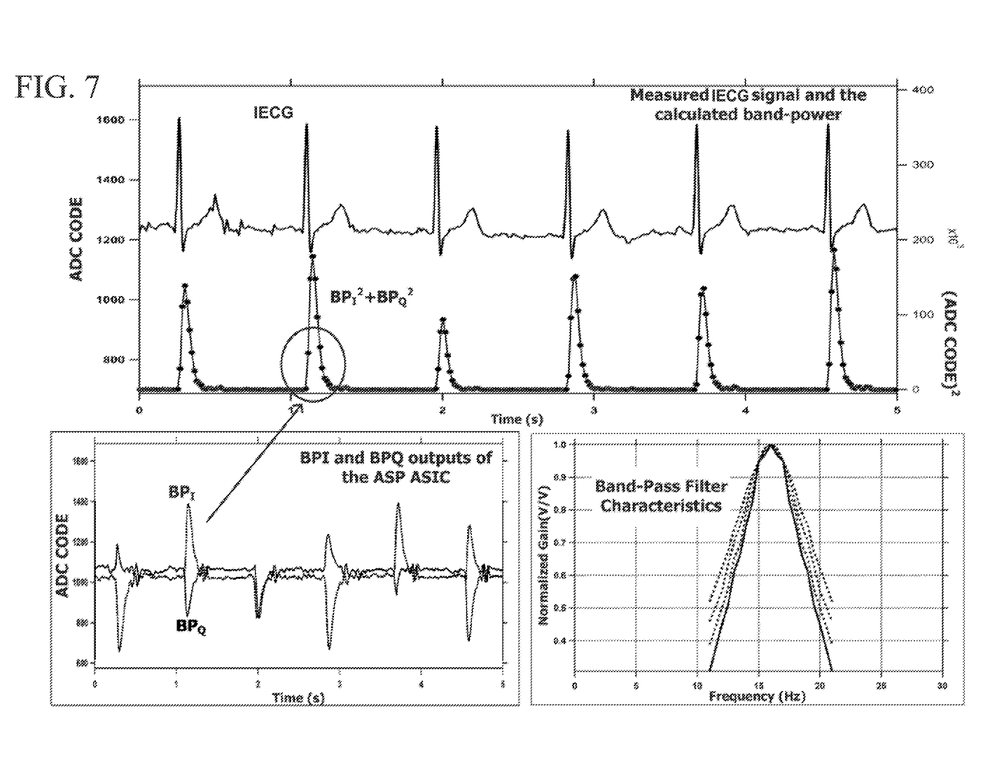

According to another exemplary embodiment of the present description, the R-peak detecting methodology shown in FIG. 1 comprises a band-pass filter with a center frequency corresponding to the frequency range of the R-wave (6 Hz-40 Hz) and the delay of the time in the center band is smaller than 10 ms.

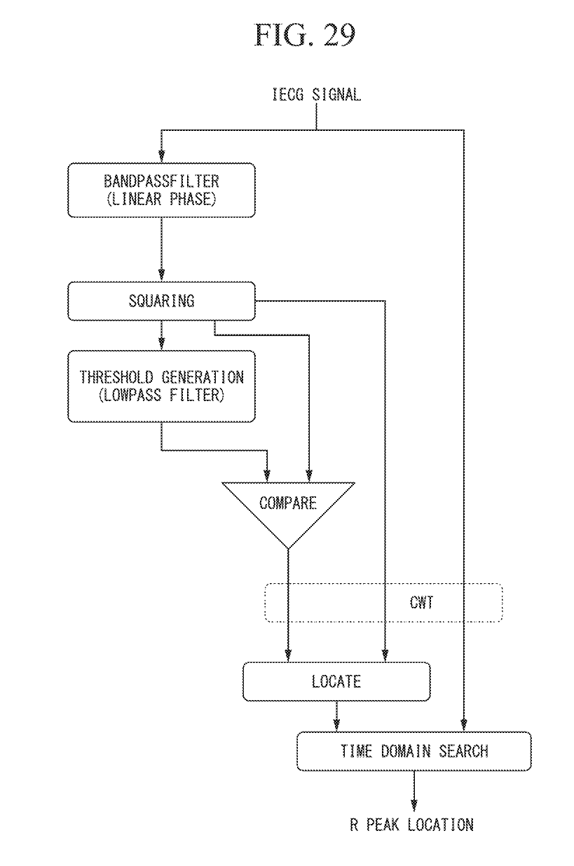

According to another exemplary embodiment of the present description, the R-peak detecting methodology shown in FIG. 29 comprises linear phase band pass filter with a center frequency of approximately 12.5 Hz.

According to another exemplary embodiment of the present description, the R-peak detecting methodology shown in FIG. 29, comprises a linear phase band pass filter in which the −3 dB compression point from the center frequency is located approximately in the range of 10 Hz-15 Hz.

According to another exemplary embodiment of the present description, the R-peak detecting methodology shown in FIG. 29 comprises a linear phase band pass filter comprising a 1st order Butterworth HPF and a 1st order Butterworth LPF.

According to another exemplary embodiment of the present description, the proposed R-peak detecting methodology may be implemented in hardware (for example, as a semiconductor chip), in software or a combination of both.

[Further Details about its Operation]

Since HPF is LEADING phase, 45 degree phase lead is expected at −3 dB compression point of is order Butterworth filter.

Since LPF is LAGGING phase, 45 degree phase lag is expected at −3 dB compression point of 1 s order Butterworth filter.

If the −3 dB compression point of the HPF and LPF is located at the same frequency, phase lag at −3 dB compression frequency should be 0 degree. Since time delay can be calculated by the product of Phase lag and reciprocal of the frequency, time delay at −3 dB compression frequency is 0 ms, too.

FIG. 15 is the MATLAB simulation result of the transfer function, in a linear phase band pass filter configuration consisting of a 1st order Butterworth HPF and 1st order Butterworth LPF. From this result, one can confirm that the delay of 12.5 Hz sinusoidal wave is less than 0 ms. Actually, slightly leading phase (time).

From FIG. 2, it is observed that the R-wave signal is spread around 6 Hz-40 Hz. But R-peak time domain search in FIG. 1 is done after filtering of the BPF. Since the BPF configuration consisting of a 1 st order Butterworth HPF and 1st order Butterworth LPF is enhancing the frequency component around 12.5 Hz, time delay around 12.5 Hz has the most importance and time delay in higher frequency can be negligible.

While preferred embodiments of the invention have been described and illustrated above, it should be understood that these are exemplary of the invention and are not to be considered as limiting. Additions, omissions, substitutions, and other modifications can be made without departing from the spirit and scope of the present invention. Accordingly, the invention is not to be considered as being limited by the foregoing description, and is only limited by the scope of the appended claims.

What is claimed is:

1. A biological signal detecting apparatus comprising:
a band pass filter (BPF) to which a biological signal is input via a lead directly connected to a subcutaneous tissue that emits the biological signal including a predetermined signal of a first frequency and which outputs a filtered biological signal by filtering a signal (biological event signal) of a predetermined frequency including the first frequency; and
a peak detecting means to which at least the filtered biological signal is input and which detects a peak location of the biological event signal by processing the filtered biological signal,
wherein the BPF includes a first order high-pass filter (HPF) which filters a frequency higher than a second frequency and a first order low-pass filter (LPF) which filters a frequency lower than a third frequency,
wherein the HPF and the LPF are connected in series between the lead and the peak detecting means, and
wherein a difference between the second frequency and the third frequency is less than or equal to 10 Hz.

2. The biological signal detecting apparatus according to claim 1,
wherein the second frequency is less than the first frequency and greater than or equal to 6 Hz, and
wherein the third frequency is greater than the first frequency and less than or equal to 25 Hz.

3. The biological signal detecting apparatus according to claim 1, further comprising:
a gain amplifier which is connected between the HPF and the LPF and amplifies a signal gain.

4. The biological signal detecting apparatus according to claim 3,
wherein the gain amplifier is a variable gain amplifier, and
wherein a gain of the variable gain amplifier is varied to an extent to which a signal component decayed by the BPF is compensated for.

5. The biological signal detecting apparatus according to claim 1, wherein both the HPF and the LPF are configured in a switched capacitor type.

6. An implantable medical device comprising:
the biological signal detecting apparatus according to claim
a diagnosis means; and
a therapy signal generating means,
wherein the biological signal is an IFCG signal,
wherein the tissue is a heart,
wherein the biological event signal is an R wave,
wherein the lead is connected to at least one of a right ventricle and a left ventricle of the heart,
wherein the diagnosis means receives an input of the peak location and outputs a diagnosis result corresponding to information of the peak location; and
wherein the therapy signal generating means receives an input of the diagnosis result and applies a therapy signal corresponding to the diagnosis result to an atrium for which therapy is necessary via the lead.
